(19) United States
(12) Patent Application Publication
(10) Pub. No.: US 2025/0384993 A1
(43) Pub. Date: Dec. 18, 2025

(54) SYSTEM ARCHITECTURE AND METHODS FOR GENERATING STERILE PROCESSING ANALYTICS

(71) Applicant: Intuitive Surgical Operations, Inc., Sunnyvale, CA (US)

(72) Inventors: Reza Khodayi Mehr, Sunnyvale, CA (US); Omid Mohareri, San Francisco, CA (US)

(73) Assignee: Intuitive Surgical Operations, Inc., Sunnyvale, CA (US)

(21) Appl. No.: 19/225,580

(22) Filed: Jun. 2, 2025

Related U.S. Application Data
(60) Provisional application No. 63/660,996, filed on Jun. 17, 2024.

(51) Int. Cl. G16H 40/20

(52) U.S. Cl. CPC G16H 40/20

(57) ABSTRACT

The arrangements disclosed herein relate to systems, apparatuses, methods, and non-transitory processor-readable media for receiving, from one or more first sensors located in a decontamination room, multi-modal data comprising three-dimensional data of at least one sterile processing (SP) procedure performed in a decontamination room, determining, using a first activity recognition machine-learning model, one or more SP actions based at least in part on the multi-modal data, determining, using an SP analysis machine-learning model, an SP metric value based at least in part on the one or more SP actions, wherein the SP metric value is indicative of at least one of efficiency or efficacy of the at least one SP procedure, and providing the SP metric value to a display device for display.

### CROSS-REFERENCE TO RELATED APPLICATION

This application claims priority to U.S. Patent Application No. 63/660,996, filed Jun. 17, 2024, the full disclosure of which is incorporated herein by reference in its entirety.

### TECHNICAL FIELD

Various embodiments disclosed herein relate to systems, apparatuses, methods, and non-transitory computer-readable media for generating sterile processing (SP) metrics based on multi-modal data collected during and for SP procedures.

### BACKGROUND

SP of medical instruments is crucial aspect in preventing healthcare-acquired infections. It has been long established that medical instruments must be properly washed, cleaned, and sterilized before being reused in order to prevent contamination and infection in a subsequent medical procedure. SP of medical instruments is usually performed at medical facilities (e.g., hospitals, out-patient facilities, etc.) Given that medical instruments are only available for reuse after SP is completed, efficiency in SP can significantly impact the overall efficiency of operations of any medical facility at which SP is performed.

### SUMMARY

Aspects of the present disclosure are directed to a system, including one or more processors, coupled with memory, to receive, from one or more first sensors located in a decontamination room, multi-modal data including three-dimensional data of at least one sterile processing (SP) procedure performed in a decontamination room, determine, using a first activity recognition machine-learning model, one or more SP actions based at least in part on the multi-modal data, determine, using an SP analysis machine-learning model, an SP metric value based at least in part on the one or more SP actions, wherein the SP metric value is indicative of at least one of efficiency or efficacy of the at least one SP procedure, and provide the SP metric value to a display device for display.

In some implementations, the one or more processors to determine, using a second activity recognition machine-learning model, one or more medical environment actions based on second multi-modal data including second three-dimensional data of a medical environment. In some implementations, the first activity recognition machine-learning model is trained using decontamination room data, and wherein the second activity recognition machine-learning model is trained using medical environment data. In some implementations, determining the one or more SP actions includes adding timestamps to the three-dimensional data associated with the one or more SP actions. In some implementations, the SP analysis machine-learning model uses as input robotic system data corresponding to medical procedures using instruments for which SP is performed. In some implementations, the robotic system data indicates usage of the instruments. In some implementations, the SP analysis machine-learning model receives the robotic system data from a robotic system in a medical environment. In some implementations, the SP analysis machine-learning model tracks the instruments from usage in the medical environment to completion of the at least one SP procedure in the decontamination room.

In some implementations, the SP metric value includes or is determined based at least in part on an SP turnaround time. In some implementations, the SP metric value includes or is determined based at least in part on SP turnaround times for each medical procedure of a plurality of medical procedures. In some implementations, the SP metric value includes or is determined based at least in part on SP turnaround times for each instrument type of a plurality of instrument types. In some implementations, the SP metric value includes one or more of SP equipment utilization, completion of steps of the at least one SP procedure, temporal metrics for the steps of the at least one SP procedure, and SP throughput. In some implementations, the one or more processors generate, using the SP analysis machine-learning model, alerts based on detecting one or more of detecting mishandled instruments, contamination of instruments, and infection risk for SP staff.

In some implementations, the one or more processors generate one or more recommendations based on one or more of the SP metric value and the three-dimensional data of the at least one SP procedure. In some implementations, the one or more recommendations include one or more of training for SP staff, decontamination room layout optimization, better resource allocation to minimize equipment idle time, and optimized staff and equipment levels. In some implementations, the display device is an interactive display. In some implementations, the display device indicates a status of instruments being processed. In some implementations, the display device includes an AR headset. In some implementations, the display device includes information, videos, images, or guidance related to performing an identified part, step, or sub-step along with an SP metric value for that part, step, or sub-part. In some implementations, the one or more first sensors include egocentric and exocentric sensors.

Aspects of the present disclosure are directed to a system, including one or more processors, coupled with memory, to receive, from one or more first sensors located in a decontamination room, multi-modal data including three-dimensional data of at least one sterile processing (SP) procedure performed in the decontamination room, wherein the SP procedure includes sterilizing an instrument used in a medical procedure performed in a medical environment or an instrument coupled to a robotic system for performing the medical procedure in the medical environment, determine, using a first activity recognition machine-learning model, one or more SP actions based at least in part on the multi-modal data, determine, using an SP analysis machine-learning model, an SP metric value for the one or more SP actions, wherein the SP metric value is determined based at least in part on a length of time of at least a portion of the one or more SP actions, and provide the SP metric value to a display device for display.

In some implementations, the one or more processors determine, using a second activity recognition machine-learning model, one or more medical environment actions based on second multi-modal data including second three-dimensional data of a medical environment. In some implementations, the first activity recognition machine-learning model is trained using decontamination room data, and wherein the second activity recognition machine-learning model is trained using medical environment data. In some implementations, the SP analysis machine-learning model uses as input robotic system data corresponding to medical procedures using instruments for which SP is performed.

In some implementations, the SP metric value includes one or more of SP equipment utilization, completion of steps of the at least one SP procedure, temporal metrics for the steps of the at least one SP procedure, and SP throughput. In some implementations, the SP analysis machine-learning model generates alerts based on detecting one or more of detecting mishandled instruments, contamination of instruments, and infection risk for SP staff. In some implementations, the one or more processors generate one or more recommendations based on one or more of the SP metric value and the three-dimensional data of the at least one SP procedure.

Aspects of the present disclosure are directed to a system, including one or more processors, coupled with memory, to receive a plurality of streams of data of a sterile processing (SP) procedure performed in a decontamination room, wherein the plurality of streams of data include three-dimensional data of the SP procedure, determine, using a machine-learning model, an SP metric value for at least a portion of the SP procedure based at least in part on the plurality of streams of data of the SP procedure, and provide the SP metric value to be displayed on a user interface.

In some implementations, the one or more processors determine, using a second machine-learning model, one or more medical environment actions based on second multi-modal data including second three-dimensional data of a medical environment. In some implementations, the machine-learning model is trained using decontamination room data, and wherein the second machine-learning model is trained using medical environment data. In some implementations, the machine-learning model uses as input robotic system data corresponding to medical procedures using instruments for which SP is performed.

In some implementations, the SP metric value includes or is determined based at least in part on an SP turnaround time. In some implementations, the SP metric value includes or is determined based at least in part on an SP turnaround time for a medical procedure of a plurality of medical procedures. In some implementations, the SP metric value includes or is determined based at least in part on an SP turnaround time for an instrument type of a plurality of instrument types. In some implementations, the SP metric value includes one or more of SP equipment utilization, completion of steps of the SP procedure, temporal metrics for the steps of the at least one SP procedure, and SP throughput. In some implementations, the machine-learning model generates alerts based on detecting one or more of detecting mishandled instruments, contamination of instruments, and infection risk for SP staff. In some implementations, the one or more processors generate one or more recommendations based on one or more of the SP metric value and the three-dimensional data of the SP procedure.

### BRIEF DESCRIPTION OF THE DRAWINGS

Various of the embodiments introduced herein may be better understood by referring to the following Detailed Description in conjunction with the accompanying drawings, in which like reference numerals indicate identical or functionally similar elements:

FIG. 1A is a schematic view of various elements appearing in a surgical theater during a surgical operation, as may occur in relation to some embodiments;

FIG. 1B is a schematic view of various elements appearing in a surgical theater during a surgical operation employing a robotic surgical system, as may occur in relation to some embodiments;

FIG. 2A is a schematic depth map rendering from an example theater-wide sensor perspective, as may be used in some embodiments;

FIG. 2B is a schematic top-down view of objects in the theater of FIG. 2A, with corresponding sensor locations;

FIG. 2C is a pair of images depicting a grid-like pattern of orthogonal rows and columns in perspective, as captured from a theater-wide visual image sensor having a rectilinear view and a theater-wide visual image sensor having a fisheye view, each of which may be used in connection with some embodiments;

FIG. 3 is a schematic representation of a series of surgical procedures within a surgical theater, their intervening nonoperative periods, and corresponding theater-wide sensor datasets for one such nonoperative period, as may occur in connection with some embodiments;

FIG. 4 is a schematic block diagram illustrating an example deployment topology for a nonoperative periods analysis system, as may be implemented in some embodiments;

FIG. 5A is a schematic representation of a collection of metrics intervals, as may be used to assess nonoperative team performance in some embodiments;

FIG. 5B is a schematic processing diagram indicating full-day relations of various intervals, including intervals from FIG. 5A, as may be applied in some embodiments;

FIG. 5C is a schematic block diagram indicating possible activity analysis class groupings, as may be used in connection with some embodiments;

FIG. 6 is a table of example task action temporal definitions, as may be used in some embodiments;

FIG. 7 is a table of additional example task action temporal definitions, as may be used in some embodiments;

FIG. 8 is a schematic block diagram illustrating various metrics and their relation in constructing a composite score (referred to as an OR analysis “ORA” score), as may be used in some embodiments;

FIG. 9A is a schematic block diagram depicting a general nonoperative analysis system processing flow, as may be implemented in some embodiments;

FIG. 9B is a schematic block diagram depicting elements in a more detailed example nonoperative analysis system processing flow than the flow depicted in FIG. 9A, as may be implemented in some embodiments;

FIG. 9C is a flow diagram illustrating various operations in an example overall process for analyzing theater-wide sensor data during nonoperative periods, as may be implemented in some embodiments;

FIG. 10 is a flow diagram illustrating various operations in an example nonoperative segment detection process, as may be performed in some embodiments;

FIG. 11A is a schematic block diagram illustrating an example information processing flow for performing object detection, as may be used in connection with some embodiments;

FIG. 11B is a flow diagram illustrating various operations in an example process for performing object detection, as may be used in some embodiments;

FIG. 12A is schematic block diagram illustrating an example object tracking information processing flow, as may be used in connection with some embodiments;

FIG. 12B is flow diagram illustrating various operations in an example process for performing object tracking, as may be used in connection with some embodiments;

FIG. 13A is a schematic visual image and depth frame theater-wide data pair, from theater-wide data video, with an indication of the optical-flow derived correspondence, as may be used in some embodiments;

FIG. 13B is a schematic top-down view of the scene depicted in FIG. 13A;

FIG. 13C is a schematic pair of visual images showing team member motion distant from and near to an imaging sensor;

FIG. 13D is a schematic top-down view depicting the team member motion presented in the visual images of FIG. 13C;

FIG. 14 is a flow diagram illustrating various operations in an example process for performing motion analysis of nonoperative periods from theater-wide data, as may be used in connection with some embodiments;

FIG. 15 is flow diagram illustrating various operations in an example process for performing clustering and outlier determination analysis based upon metric values, such as those disclosed herein, as may be performed in some embodiments;

FIG. 16 is flow diagram illustrating various operations in an example process for providing coaching feedback based upon determined metric values, as may be performed in some embodiments;

FIG. 17 is a schematic representation of GUI elements in an example dashboard interface layout for nonoperative metrics quick review, as may be implemented in some embodiments;

FIG. 18A is a schematic representation of a GUI element in an example global nonoperative metrics quick review dashboard, as may be implemented in some embodiments;

FIG. 18B is a schematic representation of arrow graphical elements, as may be used in, e.g., the element of FIG. 18A in some embodiments;

FIG. 18C is a schematic representation of an example global nonoperative metrics quick review dashboard layout, as may be implemented in some embodiments;

FIG. 19A is a plot of example interval metric values acquired in connection with an example prototype implementation of an embodiment;

FIG. 19B is a plot of example interval metric values as acquired in connection with an example prototype implementation of an embodiment;

FIG. 20 is a block diagram of an example computer system as may be used in conjunction with some of the embodiments;

FIG. 21 is a schematic block diagram illustrating an example system for generating SP metric values based on multi-modal data from a decontamination room, according to various embodiments;

FIG. 22 is a flowchart diagram illustrating an example method 2200 for generating SP metric values including efficiency or efficacy of an SP procedure based on multi-modal data from a decontamination room, according to various embodiments;

FIG. 23 is a flowchart diagram illustrating an example method 2300 for generating SP metric values based on multi-modal data from a decontamination room, according to various embodiments;

FIG. 24 is a flowchart diagram illustrating an example method 2400 for generating SP metric values based on multi-modal data from a decontamination room, according to various embodiments;

FIG. 25A is an example user interface showing instructions for an SP procedure, according to various embodiments;

FIG. 25B is an example user interface showing instructions for an SP procedure, according to various embodiments;

FIG. 26 is an example user interface showing instructions for an SP procedure, according to various embodiments;

FIG. 27 is an example user interface showing instructions for an SP procedure, according to various embodiments; and

FIG. 28 is an example user interface showing instructions for an SP procedure, according to various embodiments.

The specific examples depicted in the drawings have been selected to facilitate understanding. Consequently, the disclosed embodiments should not be restricted to the specific details in the drawings or the corresponding disclosure. For example, the drawings may not be drawn to scale, the dimensions of some elements in the figures may have been adjusted to facilitate understanding, and the operations of the embodiments associated with the flow diagrams may encompass additional, alternative, or fewer operations than those depicted here. Thus, some components and/or operations may be separated into different blocks or combined into a single block in a manner other than as depicted. The embodiments are intended to cover all modifications, equivalents, and alternatives falling within the scope of the disclosed examples, rather than limit the embodiments to the particular examples described or depicted.

### DETAILED DESCRIPTION

In medical environments (e.g., operating rooms (ORs)), surgical site infections (SSIs) can be a significant patient safety issue across the globe. For example, in the U.S., SSIs are the third most expensive type of healthcare-acquired infections, potentially costing more than $90,000 per patient. According to some studies, 2-5% of all in-patient surgery patients and 160,000-300,000 patients develop SSIs, which can lengthen patient stays for more than a week costing the U.S. health care system billions annually. SP is an integral part of preventing infection such as SSIs. The function of SP includes decontaminating, cleaning, inspecting, and sterilizing instruments (e.g., scalpels, clamps, staplers, clips, endoscopes, monopolar instruments, needle drivers, dipolar instruments, sealers, suction and irrigation instruments, etc.) used in medical procedures for future reuse. Most multi-use medical instruments must undergo SP prior to being re-used. In a medical procedure, a robotic system with specialized instruments can be used, and those specialized instruments call for specific SP procedures. Efficacy in SP for instruments and accessories (I&A) is crucial in ensuring sterility, avoiding infections, and enhancing patient outcomes. Furthermore, efficiency in SP plays a significant role in determining the overall efficiency of medical procedures and the amount of I&A inventory that is required. The duration needed for proper performance of SP of an instrument to make the instrument available for reuse is referred to as the SP turnaround time of that instrument. Faster SP turnaround time for a particular instrument means that a hospital or a medical facility can maintain less inventory for that particular instrument for a given volume of medical procedures being performed. While medical and surgical instruments may be used herein as an example of items for which SP can be performed, it should be recognized that other types of equipment, accessories, I&A, and other tools used in a medical procedure can likewise be the object for which SP is performed.

Currently, no automated systems exist that can process streams of data and provide awareness around efficacy and efficiency of SP. Existing methods for evaluating SP rely on in-person observations and manual tracking, which are inadequate and costly for computing metrics and statistically significant analytics and can be significantly inaccurate due to the observer effect. In-person observation is also difficult to scale. Expert opinion may be required to analyze collected data on SP in order to identify any inefficiency and inefficacies. Although valuable, expert opinion can be limited in scope in terms of the type of data that is available and prone to subjectivity and bias as expert opinion may be skewed by personal experience. Furthermore, there are no benchmarks on overall SP statistics across different ORs, hospitals, regions, countries, etc.

SP is a specialized task that requires extensive training, and different types and/or categories of instruments require different SP procedures. Training personnel to effectively and efficiently perform SP for various medical instruments that require different SP procedures is a difficult task, as is ensuring compliance with SP procedure instructions (e.g., instructions for use (IFUs)). Each medical instrument, or each type of medical instrument may have its own SP procedures, requiring adherence to different SP procedure instructions. Conventional and robotic medical instruments (e.g., used in robotic systems) my have different SP procedures, increasing the total number of SP procedure instructions that need to be followed by SP personnel. The turnover rate for personnel assigned to perform SP is often high, requiring continuous training and control to ensure adherence to established guidelines. In these instances, frequent monitoring, evaluation, assessment, and training (based on the result of the evaluation and assessment) can significantly improve efficacy and efficiency of the SP. However, manual monitoring and assessment is a time-consuming, labor-intensive process that is prone to human error.

In some examples, SP occurs in decontamination rooms (DRs) which include contaminated and clean sections with negative and positive air pressures, respectively, to prevent sterility breaches. For example, a clean section is filled with positive air pressure to prevent external air from flowing into the clean section, and a contaminated section is filled with negative air pressure to allow air from the clean section or another space to flow into the dirty section. DRs are often separate from medical environments where medical procedures are performed, such as operating rooms (ORs). In some examples, the DR is a space connected to the OR via a door or gate. Such door or gate can be located within a dirty section of the DR, such that the dirty section is between the clean section and the door or gate. Thus, there is a lack of continuity between use of medical instruments in medical procedures and SP of the medical instruments (e.g., different personnel may have possession of medical instruments in the OR and in the DR).

The embodiments disclosed herein can generate and provide insight and control over SP procedures by tracking, monitoring, analyzing, and optimizing SP procedures performed in the DRs. For example, systems, methods, apparatuses, and non-transitory computer-readable media are provided for tracking, analyzing, and displaying SP metrics. SP metrics can reflect the efficacy and efficiency of SP procedures, policies, and personnel, and can thus be used for evaluation and improvement of the SP.

Multi-modal environmental sensors deployed in a DR (also referred to as the first sensors) and multi-modal environmental sensors in a medical environment (ME) in which a medical procedure is performed (also referred to as the second sensors) can collect multi-modal data which is analyzed by one or more activity recognition algorithms. In some implementations, a first activity recognition algorithm analyzes first multi-modal data captured in a DR using the first sensors, and a second activity recognition algorithm analyzes second multi-modal data captured in an ME using the second sensors. The first activity recognition algorithm and in some cases both the first and second activity recognition algorithms recognize steps in SP processes and generate timestamps associated with the beginning and end of each step in the SP. ME analytics, including timestamps of steps of medical procedures and/or non-operative portions of medical procedures may be generated by a third activity recognition algorithm configured to recognize steps of medical procedures. The timestamps of the steps of the medical procedures may be correlated with the timestamps generated by the first activity recognition algorithm and the second activity recognition algorithm to determine when medical procedures end and SP begins. In some implementations, the second activity recognition algorithms identify steps performed in the ME that affect a state of medical instruments when they arrive in the DR, such as contamination levels of medical instruments and preliminary rinsing of medical instruments.

An analytics module can be configured to receive timestamps generated by robotic systems and/or to extract timestamps from robotic system data (e.g., system logs) corresponding to the instruments to be cleaned and sterilized via SP to generate SP metrics reflecting the efficiency and efficacy of the SP. For example, the robotic systems such as computer-assisted medical systems, robotically-assisted medical or surgical systems, and so on can generate the robotic system data for the operations of the robotic systems. Combining the timestamps with the robotic system data allow for tracking instruments used in medical procedures from their use by a robotic system in an ME, through SP, and back to another use by the robotic system in the ME.

The analytics module may generate the SP metrics in real time and provide real-time feedback and guidance to administrators and SP staff. The analytics module may provide interactive wall-charts showing steps of SP procedures and current progress in an SP procedure. In some implementations, an AR overlay may be provided to guide actions of SP staff in completing SP procedures.

A root cause analysis system including a root cause model can provide recommendations and alerts based on the SP metrics and events detected during SP. The recommendations may include training for SP staff, DR layout improvements, improved resource allocation to reduce equipment idle time, and optimized levels of equipment and staff.

### Example Surgical Theaters Overview

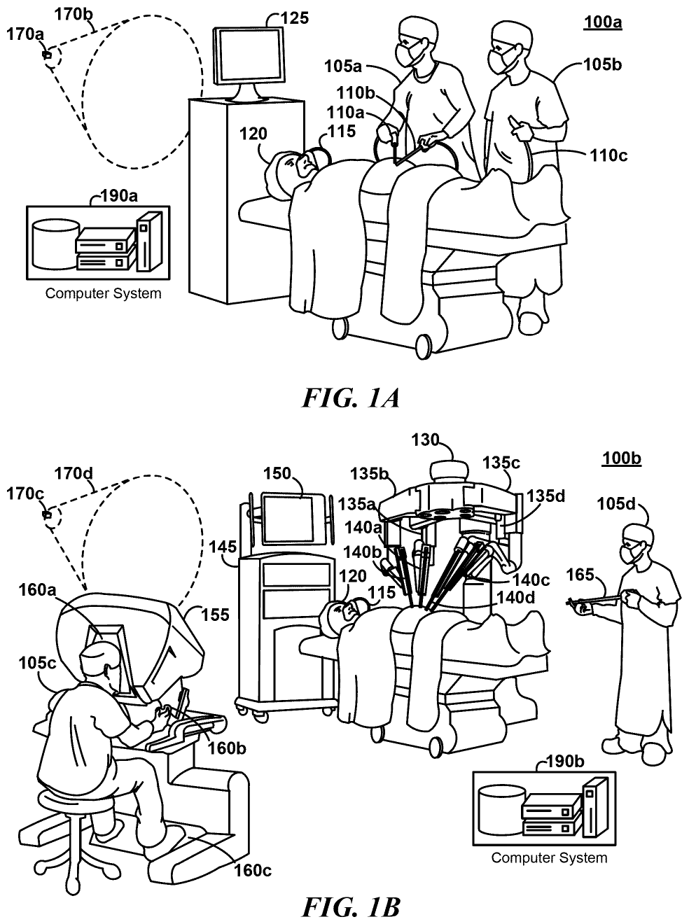

FIG. 1A is a schematic view of various elements appearing in a surgical theater 100a during a surgical operation as may occur in relation to some embodiments. Particularly, FIG. 1A depicts a non-robotic surgical theater 100a, wherein a patient-side surgeon 105a performs an operation upon a patient 120 with the assistance of one or more assisting members 105b, who may themselves be surgeons, physician's assistants, nurses, technicians, etc. The surgeon 105a may perform the operation using a variety of tools, e.g., a visualization tool 110b such as a laparoscopic ultrasound, visual image/video acquiring endoscope, etc., and a mechanical instrument 110a such as scissors, retractors, a dissector, etc.

The visualization tool 110b provides the surgeon 105a with an interior view of the patient 120, e.g., by displaying visualization output from an imaging device mechanically and electrically coupled with the visualization tool 110b. The surgeon may view the visualization output, e.g., through an eyepiece coupled with visualization tool 110b or upon a display 125 configured to receive the visualization output. For example, where the visualization tool 110b is a visual image acquiring endoscope, the visualization output may be a color or grayscale image. Display 125 may allow assisting member 105b to monitor surgeon 105a's progress during the surgery. The visualization output from visualization tool 110b may be recorded and stored for future review, e.g., using hardware or software on the visualization tool 110b itself, capturing the visualization output in parallel as it is provided to display 125, or capturing the output from display 125 once it appears on-screen, etc. While two-dimensional video capture with visualization tool 110b may be discussed extensively herein, as when visualization tool 110b is a visual image endoscope, one will appreciate that, in some embodiments, visualization tool 110b may capture three-dimensional depth data instead of, or in addition to, two-dimensional image data (e.g., with a laser rangefinder, stereoscopy, etc.).

A medical procedure (e.g., a single surgery) may include the performance of several groups (e.g., phases or stages) of actions, each group of actions forming a discrete unit referred to herein as a task. For example, locating a tumor may constitute a first task, excising the tumor a second task, and closing the surgery site a third task. Each task may include multiple actions, e.g., a tumor excision task may require several cutting actions and several cauterization actions. While some surgeries require that tasks assume a specific order (e.g., excision occurs before closure), the order and presence of some tasks in some surgeries may be allowed to vary (e.g., the elimination of a precautionary task or a reordering of excision tasks where the order has no effect). Transitioning between tasks may require the surgeon 105a to remove tools from the patient, replace tools with different tools, or introduce new tools. Some tasks may require that the visualization tool 110b be removed and repositioned relative to its position in a previous task. While some assisting members 105b may assist with surgery-related tasks, such as administering anesthesia 115 to the patient 120, assisting members 105b may also assist with these task transitions, e.g., anticipating the need for a new tool 110c.

Advances in technology have enabled procedures such as that depicted in FIG. 1A to also be performed with robotic systems, as well as the performance of procedures unable to be performed in non-robotic surgical theater 100a. Specifically, FIG. 1B is a schematic view of various elements appearing in a surgical theater 100b during a surgical operation employing a robotic surgical system, such as a da Vinci™ surgical system, as may occur in relation to some embodiments. Here, patient side cart 130 having tools 140a, 140b, 140c, and 140d attached to each of a plurality of arms 135a, 135b, 135c, and 135d, respectively, may take the position of patient-side surgeon 105a. As before, one or more of tools 140a, 140b, 140c, and 140d may include a visualization tool (here visualization tool 140d), such as a visual image endoscope, laparoscopic ultrasound, etc. An operator 105c, who may be a surgeon, may view the output of visualization tool 140d through a display 160a upon a surgeon console 155. By manipulating a hand-held input mechanism 160b and pedals 160c, the operator 105c may remotely communicate with tools 140a-d on patient side cart 130 so as to perform the surgical procedure on patient 120. Indeed, the operator 105c may or may not be in the same physical location as patient side cart 130 and patient 120 since the communication between surgeon console 155 and patient side cart 130 may occur across a telecommunication network in some embodiments. An electronics/control console 145 may also include a display 150 depicting patient vitals and/or the output of visualization tool 140d.

Similar to the task transitions of non-robotic surgical theater 100a, the surgical operation of theater 100b may require that tools 140a-d, including the visualization tool 140d, be removed or replaced for various tasks as well as new tools, e.g., new tool 165, be introduced. As before, one or more assisting members 105d may now anticipate such changes, working with operator 105c to make any necessary adjustments as the surgery progresses.

Also similar to the non-robotic surgical theater 100a, the output from the visualization tool 140d may here be recorded, e.g., at patient side cart 130, surgeon console 155, from display 150, etc. While some tools 110a, 110b, 110c in non-robotic surgical theater 100a may record additional data, such as temperature, motion, conductivity, energy levels, etc., the presence of surgeon console 155 and patient side cart 130 in theater 100b may facilitate the recordation of considerably more data than is only output from the visualization tool 140d. For example, operator 105c's manipulation of hand-held input mechanism 160b, activation of pedals 160c, eye movement with respect to display 160a, etc., may all be recorded. Similarly, patient side cart 130 may record tool activations (e.g., the application of radiative energy, closing of scissors, etc.), movement of instruments, etc., throughout the surgery. In some embodiments, the data may have been recorded using an in-theater recording device, which may capture and store sensor data locally or at a networked location (e.g., software, firmware, or hardware configured to record surgeon kinematics data, console kinematics data, instrument kinematics data, system events data, patient state data, etc., during the surgery).

Within each of theaters 100a, 100b, or in network communication with the theaters from an external location, may be computer systems 190a and 190b, respectively (in some embodiments, computer system 190b may be integrated with the robotic surgical system, rather than serving as a standalone workstation). As will be discussed in greater detail herein, the computer systems 190a and 190b may facilitate, e.g., data collection, data processing, etc.

Similarly, many of theaters 100a, 100b may include sensors placed around the theater, such as sensors 170a and 170c, respectively, configured to record activity within the surgical theater from the perspectives of their respective fields of view 170b and 170d. Sensors 170a and 170c may be, e.g., visual image sensors (e.g., color or grayscale image sensors), depth-acquiring sensors (e.g., via stereoscopically acquired visual image pairs, via time-of-flight with a laser rangefinder, structural light, etc.), or a multi-modal sensor including a combination of a visual image sensor and a depth-acquiring sensor (e.g., a red green blue depth RGB-D sensor). In some embodiments, sensors 170a and 170c may also include audio acquisition sensors or sensors specifically dedicated to audio acquisition may be placed around the theater. A plurality of such sensors may be placed within theaters 100a, 100b, possibly with overlapping fields of view and sensing range, to achieve a more holistic assessment of the surgery. For example, depth-acquiring sensors may be strategically placed around the theater so that their resulting depth frames at each moment may be consolidated into a single three-dimensional virtual element model depicting objects in the surgical theater. Examples of a three-dimensional virtual element model include a three-dimensional point cloud (also referred to as three-dimensional point cloud data). Similarly, sensors may be strategically placed in the theater to focus upon regions of interest. For example, sensors may be attached to display 125, display 150, or patient side cart 130 with fields of view focusing upon the patient 120's surgical site, attached to the walls or ceiling, etc. Similarly, sensors may be placed upon console 155 to monitor the operator 105c. Sensors may likewise be placed upon movable platforms specifically designed to facilitate orienting of the sensors in various poses within the theater.

As used herein, a “pose” refers to a position or location and an orientation of a body. For example, a pose refers to the translational position and rotational orientation of a body. For example, in a three-dimensional space, one may represent a pose with six total degrees of freedom. One will readily appreciate that poses may be represented using a variety of data structures, e.g., with matrices, with quaternions, with vectors, with combinations thereof, etc. Thus, in some situations, when there is no rotation, a pose may include only a translational component. Conversely, when there is no translation, a pose may include only a rotational component.

Similarly, for clarity, “theater-wide” sensor data refers herein to data acquired from one or more sensors configured to monitor a specific region of the theater (the region encompassing all, or a portion, of the theater) exterior to the patient, to personnel, to equipment, or to any other objects in the theater, such that the sensor can perceive the presence within, or passage through, at least a portion of the region of the patient, personnel, equipment, or other objects, throughout the surgery. Sensors so configured to collect such “theater-wide” data are referred to herein as “theater-wide sensors.” For clarity, one will appreciate that the specific region need not be rigidly fixed throughout the procedure, as, e.g., some sensors may cyclically pan their field of view so as to augment the size of the specific region, even though this may result in temporal lacunae for portions of the region in the sensor's data (lacunae which may be remedied by the coordinated panning or fields of view of other nearby sensors). Similarly, in some cases, personnel or robotics systems may be able to relocate theater-wide sensors, changing the specific region, throughout the procedure, e.g., to better capture different tasks. Accordingly, sensors 170a and 170c are theater-wide sensors configured to produce theater-wide data. “Visualization data” refers herein to visual image or depth image data captured from a sensor. Thus, visualization data may or may not be theater-wide data. For example, visualization data captured at sensors 170a and 170c is theater-wide data, whereas visualization data captured via visualization tool 140d would not be theater-wide data (for at least the reason that the data is not exterior to the patient).

### Example Theater-Wide Sensor Topologies

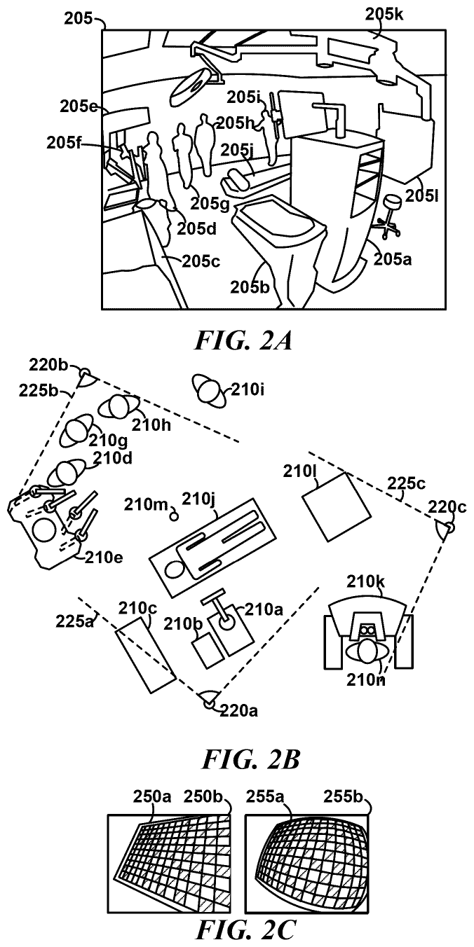

For further clarity regarding theater-wide sensor deployment, FIG. 2A is a schematic depth map rendering from an example theater-wide sensor perspective 205 as may be used in some embodiments. Specifically, this example depicts depth values corresponding to an electronics/control console 205a (e.g., the electronics/control console 145) and a nearby tray 205b, and cabinet 205c. Also within the field of view are depth values associated with a first technician 205d, presently adjusting a robotic arm (associated with depth values 205f) upon a robotic surgical system (associated with depth values 205e). Team members, with corresponding depth values 205g, 205h, and 205i, likewise appear in the field of view, as does a portion of the surgical table 205j. Depth values 205l corresponding to a movable dolly and a boom with a lighting system's depth values 205k also appear within the field of view.

The theater-wide sensor capturing the perspective 205 may be only one of several sensors placed throughout the theater. For example, FIG. 2B is a schematic top-down view of objects in the theater at a given moment during the surgical operation. Specifically, the perspective 205 may have been captured via a theater-wide sensor 220a with corresponding field of view 225a. Thus, for clarity, cabinet depth values 205c may correspond to cabinet 210c, electronics/control console depth values 205a may correspond to electronics/control console 210a, and tray depth values 205b may correspond to tray 210b. Robotic system 210e may correspond to depth values 205e, and each of the individual team members 210d, 210g, 210h, and 210i may correspond to depth values 205d, 205g, 205h, and 205i, respectively. Similarly, dolly 210l may correspond to depth values 205l. Depth values 205j may correspond to table 210j (with an outline of a patient shown here for clarity, though the patient has not yet been placed upon the table corresponding to depth values 205j in the example perspective 205). A top-down representation of the boom corresponding to depth values 205k is not shown for clarity, though one will appreciate that the boom may likewise be considered in various embodiments.

As indicated, each of the sensors 220a, 220b, 220c is associated with different fields of view 225a, 225b, and 225c, respectively. The fields of view 225a-c may sometimes have complementary characters, providing different perspectives of the same object, or providing a view of an object from one perspective when it is outside, or occluded within, another perspective. Complementarity between the perspectives may be dynamic both spatially and temporally. Such dynamic character may result from movement of an object being tracked, but also from movement of intervening occluding objects (and, in some cases, movement of the sensors themselves). For example, at the moment depicted in FIGS. 2A and 2B, the field of view 225a has only a limited view of the table 210j, as the electronics/control console 210a substantially occludes that portion of the field of view 225a. Consequently, in the depicted moment, the field of view 225b is better able to view the surgical table 210j. However, neither field of view 225b nor 225a has an adequate view of the operator 210n in console 210k. To observe the operator 210n (e.g., when they remove their head in accordance with “head out” events), field of view 225c may be more suitable. However, over the course of the data capture, these complementary relationships may change. For example, before the procedure begins, electronics/control console 210a may be removed and the robotic system 210e moved into the position 210m. In this configuration, field of view 225a may instead be much better suited for viewing the patient table 210j than the field of view 225b. As another example, movement of the console 210k to the presently depicted pose of electronics/control console 210a may render field of view 225a more suitable for viewing operator 210n, than field of view 225c. Suitability of a field of view may thus depend upon the number and duration of occlusions, quality of the field of view (e.g., how close the object of interest is to the sensor), and movement of the object of interest within the theater. Such changes may be transitory and short in duration, as when a team member moving in the theater briefly occludes a sensor, or they may be chronic or sustained, as when equipment is moved into a fixed position throughout the duration of the procedure.

As mentioned, the theater-wide sensors may take a variety of forms and may, e.g., be configured to acquire visual image data, depth data, both visual and depth data, etc. One will appreciate that visual and depth image captures may likewise take on a variety of forms, e.g., to afford increased visibility of different portions of the theater. For example, FIG. 2C is a pair of images 250b, 255b depicting a grid-like pattern of orthogonal rows and columns in perspective, as captured from a theater-wide sensor having a rectilinear view and a theater-wide sensor having a fisheye view, respectively. More specifically, some theater-wide sensors may capture rectilinear visual images or rectilinear depth frames, e.g., via appropriate lenses, post-processing, combinations of lenses and post-processing, etc. while other theater-wide sensors may instead, e.g., acquire fisheye or distorted visual images or rectilinear depth frames, via appropriate lenses, post-processing, combinations of lenses and post-processing, etc. For clarity, image 250b depicts a checkboard pattern in perspective from a rectilinear theater wide sensor. Accordingly, the orthogonal rows and columns 250a shown here in perspective, retain linear relations with their vanishing points. In contrast, image 255b depicts the same checkboard pattern in the same perspective, but from a fish-eye theater-wise sensor perspective. Accordingly, the orthogonal rows and columns 255a, while in reality retaining a linear relationship with their vanishing points (as they appear in image 250b) appear here from the sensor data as having curved relations with their vanishing points. Thus, each type of sensor, and other sensor types, may be used alone, or in some instances, in combination, in connection with various embodiments.

Similarly, one will appreciate that not all sensors may acquire perfectly rectilinear, fisheye, or other desired mappings. Accordingly, checkered patterns, or other calibration fiducials (such as known shapes for depth systems), may facilitate determination of a given theater-wide sensor's intrinsic parameters. For example, the focal point of the fisheye lens, and other details of the theater-wide sensor (principal points, distortion coefficients, etc.), may vary between devices and even across the same device over time. Thus, it may be necessary to recalibrate various processing methods for the particular device at issue, anticipating the device variation when training and configuring a system for machine learning tasks. Additionally, one will appreciate that the rectilinear view may be achieved by undistorting the fisheye view once the intrinsic parameters of the camera are known (which may be useful, e.g., to normalize disparate sensor systems to a similar form recognized by a machine learning architecture). Thus, while a fisheye view may allow the system and users to more readily perceive a wider field of view than in the case of the rectilinear perspective, when a processing system is considering data from some sensors acquiring undistorted perspectives and other sensors acquiring distorted perspectives, the differing perspectives may be normalized to a common perspective form (e.g., mapping all the rectilinear data to a fisheye representation or vice versa).

### Example Surgical Theater Nonoperative Data

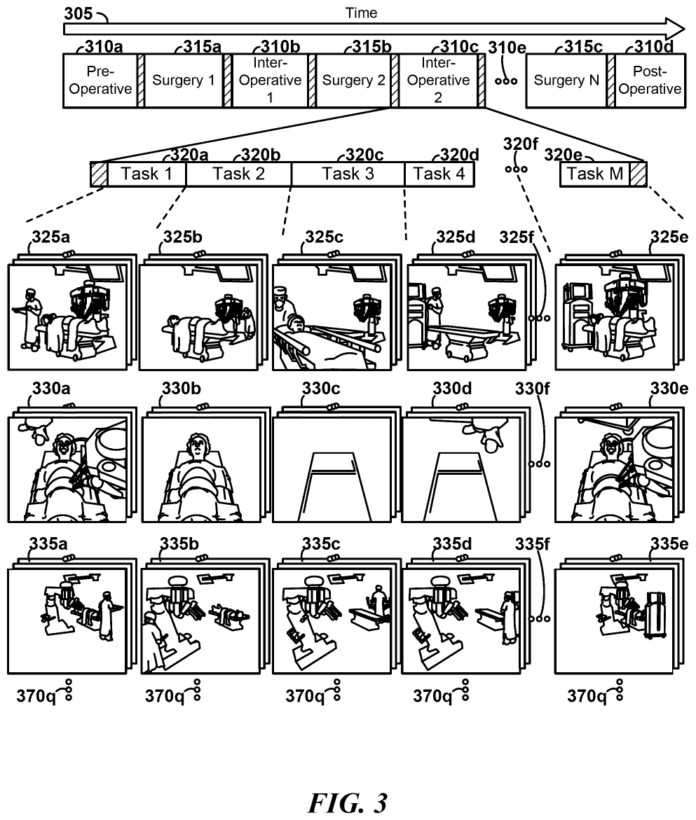

As discussed above, granular and meaningful assessment of team member actions and performance during nonoperative periods in a theater may reveal opportunities to improve efficiency and to avoid inefficient behavior having the potential to affect downstream operative and nonoperative periods. For context, FIG. 3 depicts a state of a single operating room over time 305, e.g., over the course of a day. In this example, during an initial pre-surgical period 310a, the team may prepare the operating room for the day's procedures, collecting appropriate equipment, reviewing scheduled tasks, etc. After performing the day's first surgery 315a, a nonoperative inter-operative period 310b will follow wherein the team performs the turnover from the operating room configuration for performing the surgery 315a to the configuration for performing the surgery 315b. Such alternating nonoperative and operative periods may continue throughout the day, e.g., nonoperative inter-surgical period 310c here follows the second surgery 315b, etc. After the final procedure 315c is performed for the day, the team may perform any final maintenance operations, may secure and put away equipment, deactivate devices, upload data, etc., during the post-operative period 310d. Ellipsis 310e indicates the possibility of additional intervening operative and nonoperative states (though, naturally, in some theaters there may instead by only one surgery during the day). Because of the theater operations' sequential character, an error in an upstream period can cause errors and delays to cascade through downstream periods. For example, improper alignment of equipment during pre-surgical period 310a may result in a delay during surgery 315a. This delay may itself require nonoperative period 310b to be shortened, providing a team member insufficient time to perform proper cleaning procedures, thereby placing the patient of surgery 315b's health at risk. Thus, inefficiencies early in the day may result in the delay, poor execution, or rescheduling of downstream actions. Conversely, efficiencies early in the day may provide tolerance downstream for unexpected events, facilitating more predictable operation outcomes and other benefits.

Each of the theater states, including both the operative periods 315a, 315b, etc. and nonoperative periods 310a, 310b, 310c, 310d, etc. may be divided into a collection of tasks. For example, the nonoperative period 310c may be divided into the tasks 320a, 320b, 320c, 320d, and 320e (with intervening tasks represented by ellipsis 320f). In this example, at least three theater-wide sensors were present in the OR, each sensor capturing at least visual image data (though one will appreciate that there may be fewer than three streams, or more, as indicated by ellipses 370q). Specifically, a first theater-wide sensor captured a collection of visual images 325a (e.g., visual image video) during the first nonoperative task 320a, a collection of visual images 325b during the second nonoperative task 320b, a collection of visual images 325c during the third nonoperative task 320c, a collection of visual images 325d during the fourth nonoperative task 320d, and the collection of visual images 325e during the last nonoperative task 320e (again, intervening groups of frames may have been acquired for other tasks as indicated by ellipsis 325f).

Contemporaneously during each of the tasks of the second nonoperative period 310c, the second theater-wide sensor may acquire the data collections 330a-e (ellipsis 330f depicting possible intervening collections), and the third theater-wide sensor may acquire the collections of 335a-e (ellipsis 335f depicting possible intervening collections). Thus, one will appreciate, e.g., that the data in sets 325a, 330a, and 335a may be acquired contemporaneously by the three theater-wide sensors during the task 320a (and, similarly, each of the other columns of collected data associated with each respective nonoperative task). Again, though visual images are shown in this example, one will appreciate that other data, such as depth frames, may alternatively, or additionally, be likewise acquired in each collection.

Thus, in task 320a, which may be an initial “cleaning” task following the surgery 315b, the sensor associated with collections 325a-e depicts a team member and the patient in a first perceptive. In contrast, the sensor capturing collections 335a-e is located on the opposite side of the theater and provides a fisheye view from a different perspective. Consequently, the second sensor's perception of the patient is more limited. The sensor associated with collections 330a-e is focused upon the patient, however, this sensor's perspective doesn't depict the team member very well in the collection 330a, whereas the collection 325a does provide a clear view of the team member.

Similarly, in task 320b, which may be a “roll-back” task, moving the robotic system away from the patient, the theater-wide sensor associated with collections 330a-e depicts that the patient is no longer subject to anesthesia, but does not depict the state of the team member relocating the robotic system. Rather, the collections 325b and 335b each depict the team member and the new pose of the robotic system at a point distant from the patient and operating table (though the sensor associated with the stream collections 335a-e is better positioned to observe the robot in its post-rollback pose).

In task 320c, which may be a “turnover” or “patient out” task, a team member escorts the patient out of the operating room. While the theater-wide sensor associated with collection 325c has a clear view of the departing patient, the theater-wide sensor associated with the collection 335c may be too far away to observe the departure in detail. Similarly, the collection 330c only indicates that the patient is no longer on the operating table.

In task 320d, which may be a “setup” task, a team member positions equipment which will be used in the next operative period (e.g., the final surgery 315c if there are no intervening periods in the ellipsis 310e).

Finally, in task 320e, which may be a “sterile prep” task before the initial port placements and beginning of the next surgery (again, e.g., surgery 315c), the theater-wide sensor associated with collection 330e is able to perceive the pose of the robotic system and its arms, as well as the state of the new patient. Conversely, collections 325e and 335e may provide wider contextual information regarding the state of the theater.

Thus, one can appreciate the holistic benefit of multiple sensor perspectives, as the combined views of the streams 325a-e, 330a-e, and 335a-e may provide overlapping situational awareness. Again, as mentioned, not all of the sensors may acquire data in exactly the same manner. For example, the sensor associated with collections 335a-e may acquire data from a fisheye perspective, whereas the sensors associated with collections 325a-e and 330a-e may acquire rectilinear data. Similarly, there may be fewer or more theater-wide sensors and streams than are depicted here. Generally, because each collection is timestamped, it will be possible for a reviewing system to correlate respective streams' representations, even when they are of disparate forms. Thus, data directed to different theater regions may be reconciled and reviewed. Unfortunately, as mentioned, unlike periods 315a-c, surgical instruments, robotic systems, etc., may no longer be capturing data during the nonoperative periods (e.g., periods 310a-d). Accordingly, systems and reviewers regularly accustomed to analyzing the copious datasets available from periods 315a-c may find it especially difficult to review the more sparse data of periods 310a-d as they may need to rely only upon the disparate theater-wide streams 325a-e, 330a-e, and 335a-e. Even as the reader may have perceived in considering this figure, manually reconciling disparate, but contemporaneously captured perspectives, may be cognitively taxing upon a human reviewer.

### Example Nonoperative Activity Data Processing Overview

Various embodiments employ a processing pipeline facilitating analysis of nonoperative periods, and may include methods to facilitate iterative improvement of the surgical team's performance during these periods. Particularly, some embodiments include computer systems configured to automatically measure and analyze nonoperative activities in surgical operating rooms and recommend customized actionable feedback to operating room staff or hospital management based upon historical dataset patterns so as, e.g., to improve workflow efficiency. Such systems can also help hospital management assess the impact of new personnel, equipment, facilities, etc., as well as scale their review to a larger number, and more disparate types, of surgical theaters and surgeries, consequently driving down workflow variability. As discussed, various embodiments may be applied to surgical theaters having more than one modality, e.g., robotic, non-robotic laparoscopic, non-robotic open. Neither are various of the disclosed approaches limited to nonoperative periods associated with specific types of surgical procedures (e.g., prostatectomy, cholecystectomy, etc.).

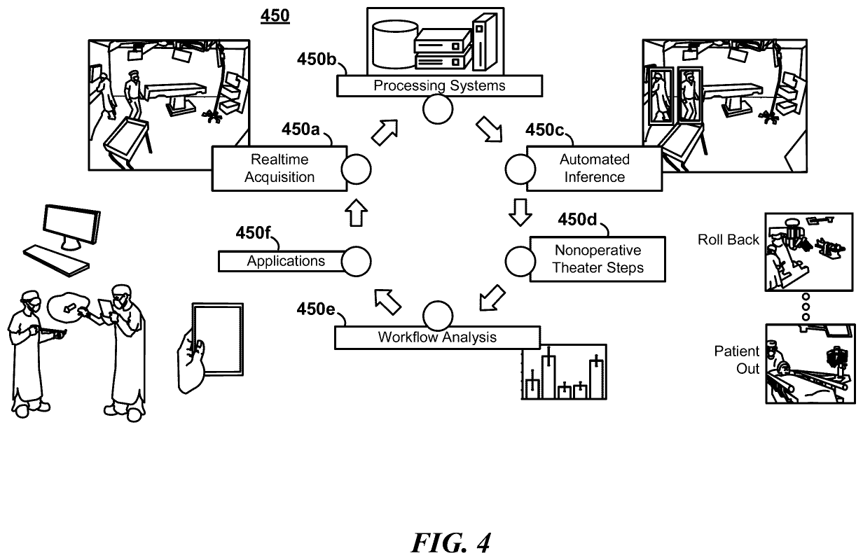

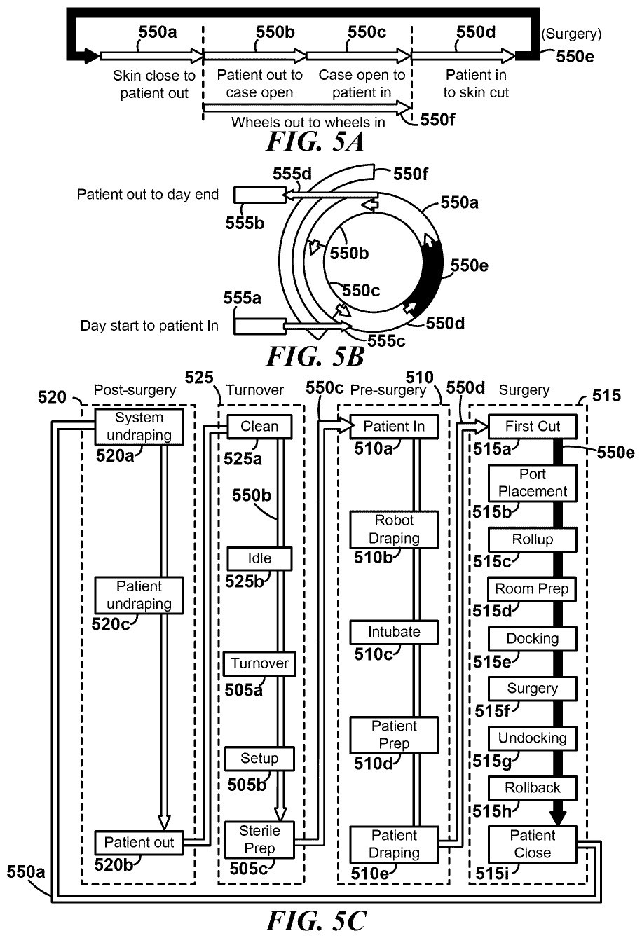

FIG. 4 is a schematic block diagram illustrating an example deployment topology 450 for a nonoperative periods analysis system of certain embodiments. As described herein, during realtime acquisition 450a, data may be collected from one or more theater-wide sensors in one or more perspectives. Multimodal (e.g., visual image and depth) sensor suites within a surgical theater (whether robotic or non-robotic) produce a wide variety of data. Consolidating this data into elemental and composite OR metrics, as described herein, may more readily facilitate analysis. To determine these metrics, the data may be provided to a processing systems 450b, described in greater detail herein, to perform automated inference 450c, including the detection of objects in the theater, such as personnel and equipment, as well as to segment the theater-wide data into distinct steps 450d (which may, e.g., correspond to the groupings and their respective actions discussed herein with respect to FIGS. 5A-C). The discretization of the theater-wide data into the steps 450d may facilitate more meaningful and granular determinations of metrics from the theater-wide data via various workflow analytics 450e, e.g., to ascertain surgical theater efficiency, to provide actionable coaching recommendations, etc.

Following the generation of such metrics during workflow analysis 450e, embodiments also disclose software and algorithms for presentation of the metric values along with other suitable information to users (e.g., consultants, students, medical staff, and so on) and for outlier detection within the metric values relative to historical patterns. As used herein, information of a plurality of medical procedures (e.g., procedure-related information, case-related information, information related to medical environments such as the ORs, and so on) refers to metric values and other associated information determined in the manners described herein. These analytics results may then be used to provide coaching and feedback via various applications 450f. Software applications 450f may present various metrics and derived analysis disclosed herein in various interfaces as part of the actionable feedback, a more rigorous and comprehensive solution than the prior use of human reviewers alone. One will appreciate that such applications 450f may be provided upon any suitable computer system, including desktop applications, tablets, augmented reality devices, etc. Such computer system can be located remote from the surgical theaters 100a and 100b in some examples. In other examples, such computer system can be located within the surgical theaters 100a and 100b (e.g., within the OR or the medical facility in which the hospital or OR processes occur). In one example, a consultant can review the information of a plurality of medical procedures via the applications 450f to provide feedback. In another example, a student can review the information of a plurality of medical procedures via the applications 450f to improve learning experience and to provide feedback. This feedback may result in the adjustment of the theater operation such that subsequent application of the steps 450a-f identify new or more subtle inefficiencies in the team's workflow. Thus, the cycle may continue again, such that the iterative, automated OR workflow analytics facilitate gradual improvement in the team's performance, allowing the team to adapt contextually based on upon the respective adjustments. Such iterative application may also help reviewers to better track the impact of the feedback to the team, analyze the effect of changes to the theater composition and scheduling, as well as for the system to consider historical patterns in future assessments and metrics generation.

### Example Nonoperative Interval Divisions

FIG. 5A is a schematic representation of a collection of metrics intervals as may be used to assess nonoperative team performance in some embodiments. One will appreciate that the intervals may be applied cyclically in accordance with the alternating character of the operative and nonoperative periods in the theater described above in FIG. 3. For example, initially, the surgical operation 315b may correspond to the interval 550e. Following the operation 315b's completion, actions and corresponding data in the theater may be allocated to consecutive intervals 550a-d during the subsequent nonoperative period 310c. Data and actions in the next surgery (e.g., surgery 315c, if there are no intervening periods in ellipsis 310e), may then be ascribed again to a second instance of the interval 550e, and so forth (consequently, data from each of the nonoperative periods 310b, 310b will be allocated to instances of intervals 550a-d). Intervals may also be grouped into larger intervals, as is the case here with the “wheels out to wheels in” interval 550f, which groups the intervals 550b and 550c, sharing the start time of interval 550b and the end time of interval 550c. Consolidating theater-wide data into this taxonomy, in conjunction with various other operations disclosed herein, may more readily facilitate analysis in a manner amenable to larger efficiency review, as described in greater detail herein. For example, organizing data in this manner may facilitate comparisons with different days of the week over the course of the month across theaters, surgery configurations (both robotic and non-robotic), and teams, with specific emphasis upon particular of these intervals 550a-d appearing in the corresponding nonoperative periods. Though not part of the nonoperative period, in some embodiments, it may still be useful to determine the duration of the surgery in interval 550e, as the duration may inform the efficiency or inefficiency of the preceding or succeeding nonoperative period. Accordingly, in some embodiments, some of the disclosed metrics may consider events and actions in this interval 550e, even when seeking ultimately to assess the efficiency of a nonoperative period.

For further clarity in the reader's understanding, FIG. 5B is a schematic block diagram indicating full-day relations of the elements from FIG. 5A. Specifically, as discussed above, instances of the intervals of FIG. 5A may be created cyclically in accordance with the alternating operative and nonoperative periods of FIG. 3. In some embodiments, when considering full day data (e.g., data including the nonoperative pre-operative period 310a, nonoperative post-operative period 310d, and all intervening periods), the system may accordingly anticipate a preliminary interval “day start to patient in” 555a to account for actions within the pre-operative period 310a. This interval may, e.g., begin when the first personnel enters the theater for the day and may end when the patient enters the theater for the first surgery. Accordingly, as shown by the arrow 555c, this may result in a transition to the first instance of the “patient in to skin cut” interval 550d. From there, as indicated by the circular relation, the data may be cyclically grouped into instances of the intervals 550a-e, e.g., in accordance with the alternating periods 315a, 310b, 315b, 310c. etc. until the period 315c.

At the conclusion of the final surgery for the day (e.g., surgery 315c), and following the last instance of the interval 550a after that surgery, then rather than continue with additional cyclical data allocations among instances of the intervals 550a-e, the system may instead transition to a final “patient out to day end” interval 555b, as shown by the arrow 555d (which may be used to assess nonoperative post-operative period 310d). The “patient out to day end” interval 555b may end when the last team member leaves the theater or the data acquisition concludes. One will appreciate that various of the disclosed computer systems may be trained to distinguish actions in the interval 555b from the corresponding data of interval 550b (naturally, conclusion of the data stream may also be used in some embodiments to infer the presence of interval 555b). Though concluding the day's actions, analysis of interval 555b may still be appropriate in some embodiments, as actions taken at the end of one day may affect the following day's performance.

### Example Task to Interval Assignments and Action Temporal Intervals

In some embodiments, the durations of each of intervals 550a-e may be determined based upon respective start and end times of various tasks or actions within the theater. Naturally, when the intervals 550a-e are used consecutively, the end time for a preceding interval (e.g., the end of interval 550c) may be the start time of the succeeding interval (e.g., the beginning of interval 550d). When coupled with a task action grouping ontology, theater-wide data may be readily grouped into meaningful divisions for downstream analysis. This may facilitate, e.g., consistency in verifying that team members have been adhering to proposed feedback, as well as computer-based verification of the same, across disparate theaters, team configurations, etc. As will be explained, some task actions may occur over a period of time (e.g., cleaning), while others may occur at a specific moment (e.g., entrance of a team member).

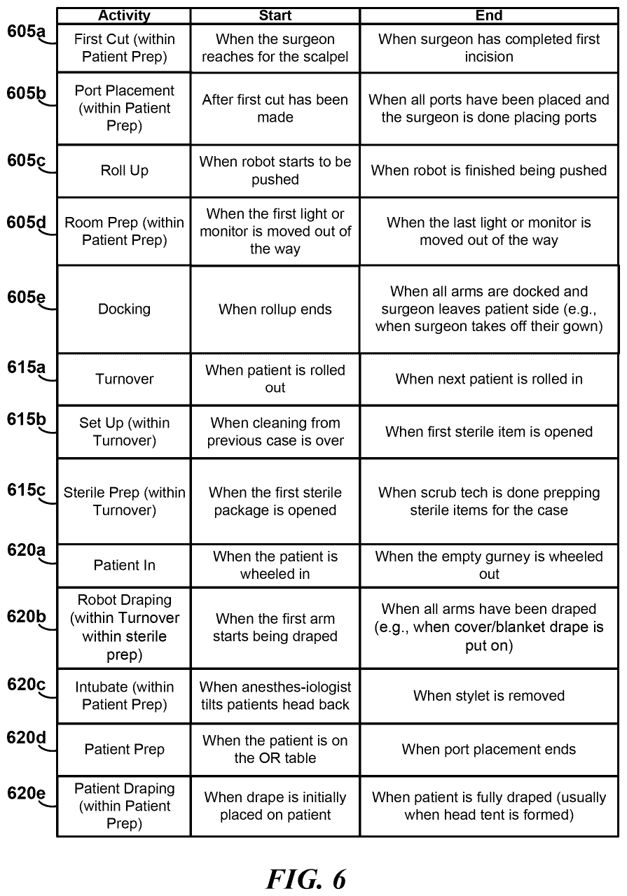

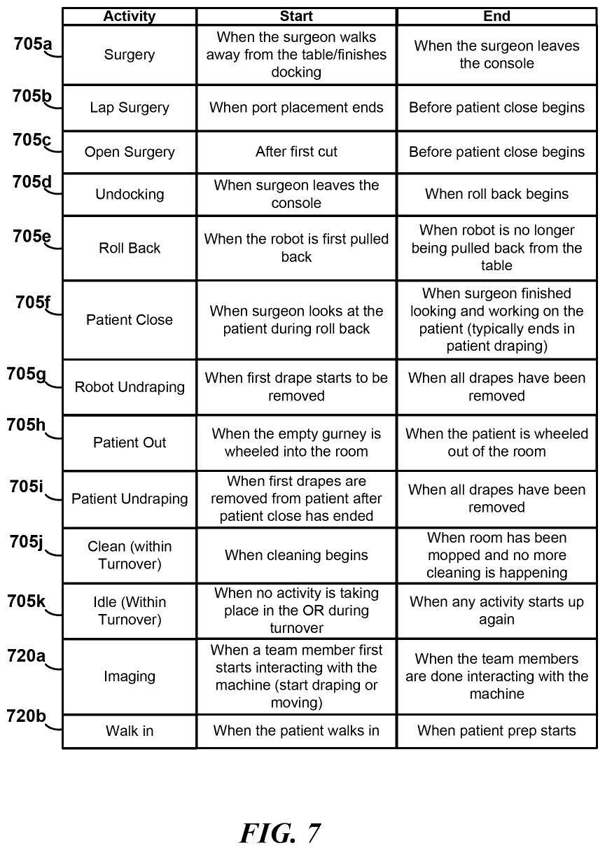

Specifically, FIG. 5C depicts four high-level task action classes or groupings of tasks, referred to for example as phases or stages: post-surgery 520, turnover 525, pre-surgery 510, and surgery 515. Surgery 515 may include the tasks or actions 515a-i. As will be discussed, FIGS. 6 and 7 provide various example temporal definitions for the actions, though for the reader's appreciation, brief summaries will be provided here. Specifically, the task “first cut” 515a, may correspond to a time when the first incision upon the patient occurs (consider, e.g., the duration 605a). The task “port placement” 515b, may correspond to a duration between the time when a first port is placed into the patient and the time when the last port is placed (consider, e.g., the duration 605b). The task “rollup” 515c, may correspond to the duration in which a team member begins moving a robotic system to a time when the robotic system assumes the pose it will use during at least an initial portion of the surgical procedure (consider, e.g., the duration 605c). The task “room prep” 515d, may correspond to a duration beginning with the first surgery preparation action specific to the surgery being performed and may conclude with the last preparation action specific to the surgery being performed (consider, e.g., the duration 605d). The task “docking” 515e, may correspond to a duration starting when a team member begins docking a robotic system and concludes when the robotic system is docked (consider, e.g., the duration 605e). The task “surgery” 515f, may correspond with a duration starting with the first incision and ending with the final closure of the patient (consider, e.g., the durations 705a-c for respective contemplated surgeries, specifically the robotic surgery 705a and non-robotic surgeries 705b and 705c). Naturally, in many taxonomies, these action blocks may be further broken down into considerably more action and task divisions in accordance with the analyst's desired focus (e.g., if the action “port placement” 515b were associated with an inefficiency, a supplemental taxonomy wherein each port's placement were a distinct action, with its own measured duration, may be appropriate for refining the analysis). Here, however, as nonoperative period actions are the subject of review, the general task “surgery” 515f (e.g., one of durations 705a-c) may suffice, despite surgery's encompassing many constituent actions. The task “undocking” 515g, may correspond to a duration beginning when a team member starts to undock a robotic system and concludes when the robotic system is undocked (consider, e.g., the duration 705d). The task “rollback” 515h, may correspond to a duration when a team member begins moving a robotic system away from a patient and concludes when the robotic system assumes a pose it will retain until turnover begins (consider, e.g., the duration 705e). The task “patient close” 515a, may correspond to a duration (e.g., duration 705f) when the surgeon observes the patient during rollback (e.g., one will appreciate by this example that some action durations may overlap and proceed in parallel).

Within the post-surgical class grouping 520, the task “robot undraping” 520a may correspond to a duration when a team member first begins undraping a robotic system and ends when the robotic system is undraped (consider, e.g., the duration 705g). The task “patient out” 520b, may correspond to a time, or duration, during which the patient leaves the theater (consider, e.g., the duration 705h). The task “patient undraping” 520c, may correspond to a duration beginning when a team member begins undraping the patient and ends when the patient is undraped (consider, e.g., the duration 705i).

Within the turnover class grouping 525, the task “clean” 525a, may correspond to a duration starting when the first team member begins cleaning equipment in the theater and concludes when the last team member (which may be the same team member) completes the last cleaning of any equipment (consider, e.g., the duration 705j). The task “idle” 525b, may correspond to a duration that starts when team members are not performing any other task and concludes when they begin performing another task (consider, e.g., the duration 705k). The task “turnover” 505a may correspond to a duration that starts when the first team member begins resetting the theater from the last procedure and concludes when the last team member (which may be the same team member) finishes the reset (consider, e.g., the duration 615a). The task “setup” 505b may correspond to a duration that starts when the first team member begins changing the pose of equipment to be used in a surgery, and concludes when the last team member (which may be the same team member) finishes the last equipment pose adjustment (consider, e.g., the duration 615a). The task “sterile prep” 505c, may correspond to a duration that starts when the first team member begins cleaning the surgical area and concludes when the last team member (which may be the same team member) finishes cleaning the surgical area (consider, e.g., the duration 615c). Again, while shown here in linear sequences, one will appreciate that task actions within the classes may proceed in orders other than that shown or, in some instances, may refer to temporal periods which may overlap and may proceed in parallel (e.g., when performed by different team members).

Within pre-surgery class grouping 510, the task “patient in” 510a may correspond to a duration that starts and ends when the patient first enters the theater (consider, e.g., the duration 620a). The task “robot draping” 510b may correspond to a duration that starts when a team member begins draping the robotic system and concludes when draping is complete (consider, e.g., the duration 620b). The task “intubate” 510c may correspond to a duration that starts when intubation of the patient begins and concludes when intubation is complete (consider, e.g., the duration 620c). The task “patient prep” 510d may correspond to a duration that starts when a team member begins preparing the patient for surgery and concludes when preparations are complete (consider, e.g., the duration 620d). The task “patient draping” 510e may correspond to a duration that starts when a team member begins draping the patient and concludes when the patient is draped (consider, e.g., the duration 620e).

Though not discussed herein, as mentioned, one will appreciate the possibility of additional or different task actions. For example, the durations of “Imaging” 720a and “Walk In” 720b, though not part of the example taxonomy of FIG. 5C, may also be determined in some embodiments.

Thus, as indicated by the respective arrows in FIG. 5C, the intervals of FIG. 5A may be allocated as follows. “Skin-close to patient-out” 550a may begin at the last closing operation 515j of the previous surgery interval and concludes with the patient's departure from the theater (e.g., from the end of the last suture at block 515i until the patient has departed at block 520b). Similarly, the interval “Patient-out to case-open” 550b may begin when the patient's departure from the theater at block 520b and concludes with the start of sterile prep at block 505c for the next case.

The interval “case-open to patient-in” 550c, may begin with the start of the sterile prep at block 505c and conclude with the start of the new patient entering the theater at block 510a. The interval “patient-in to skin cut” 550d may begin when the new patient enters the theater at block 510a and concludes at the start of the first cut at block 515. The surgery itself may occur during the interval 550e as shown.

As previously discussed, the “wheels out to wheels in” interval 550f is the interval from the start of “Patient out to case open” 550b and concludes with the end of “case open to patient in” 550c.

### Example Nonoperative Metric Generation and Scoring

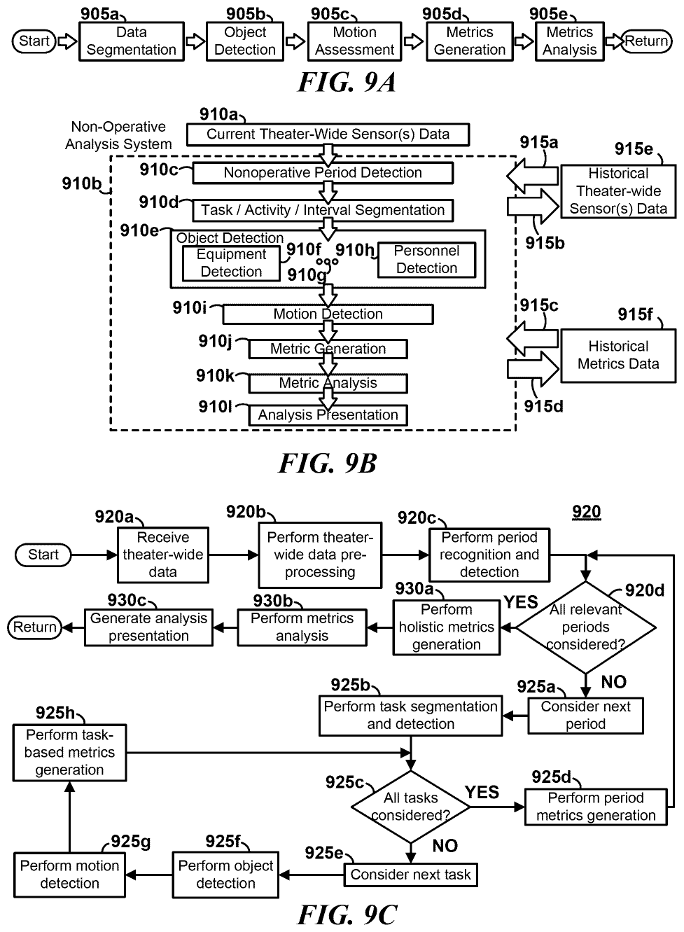

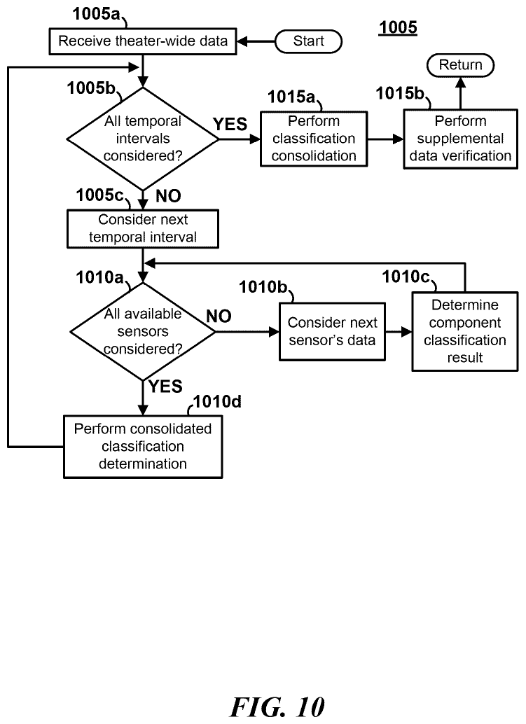

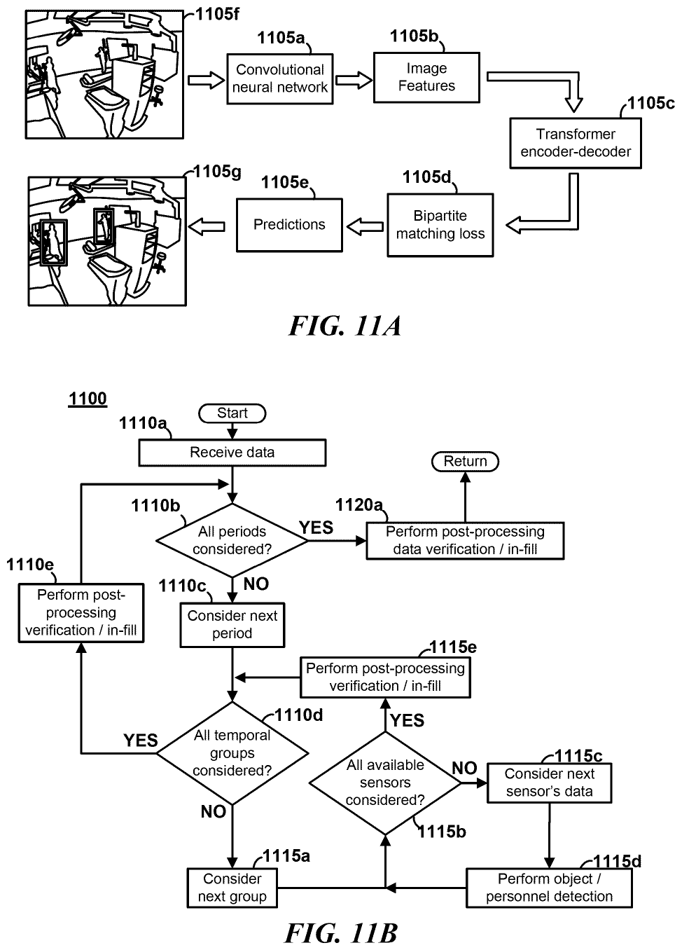

After the nonoperative segments have been identified (e.g., using systems and methods discussed herein with respect to FIGS. 9A-C and FIG. 10), the number and location of objects (e.g., using systems and methods discussed herein with respect to FIGS. 9A-C and FIGS. 11A-B), such as personnel, within each segment, and their respective motions have been identified (e.g., using systems and methods discussed herein with respect to FIGS. 9A-C, 12A-B, 13A-D, and 14), the system may generate one or more metric values. As mentioned, the duration and relative times of the intervals, classes, and task actions of FIGS. 5A-C may themselves serve as metrics.

Various embodiments may also determine “composite” metric scores based upon various of the other determined metrics. These metrics assume the functional form of EQN. 1:

$$s = f ( m ) \qquad ( 1 )$$

where s refers to the composite metric score value, which may be confined to a range, e.g., from 0 to 1, from 0 to 100, etc., and f(⋅) represents the mapping from individual metrics to the composite score. For example, m may be a vector of metrics computed using various data streams and models as disclosed herein. In such composite scores, in some embodiments, the constituent metrics may fall within one of temporal workflow, scheduling, human resource, or other groupings disclosed herein.

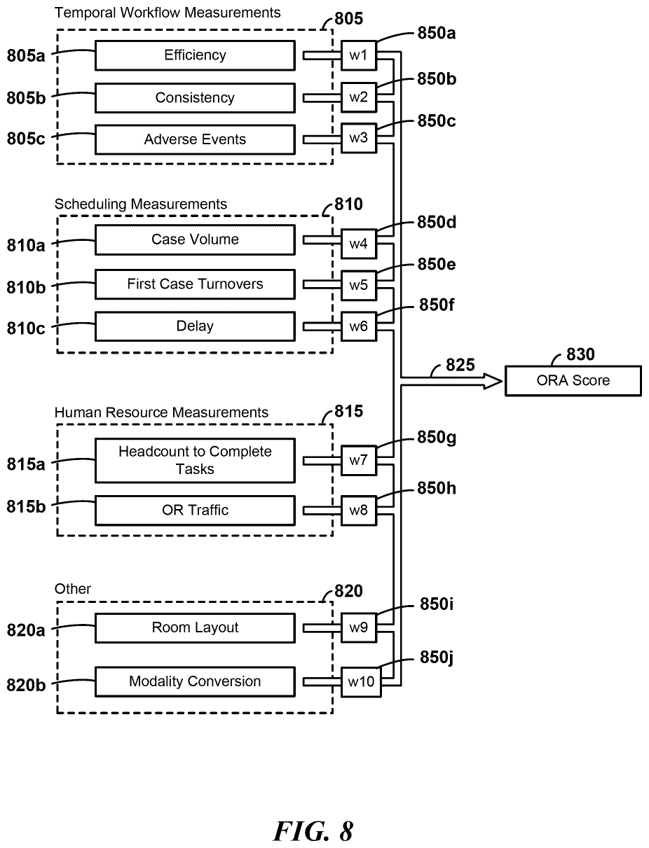

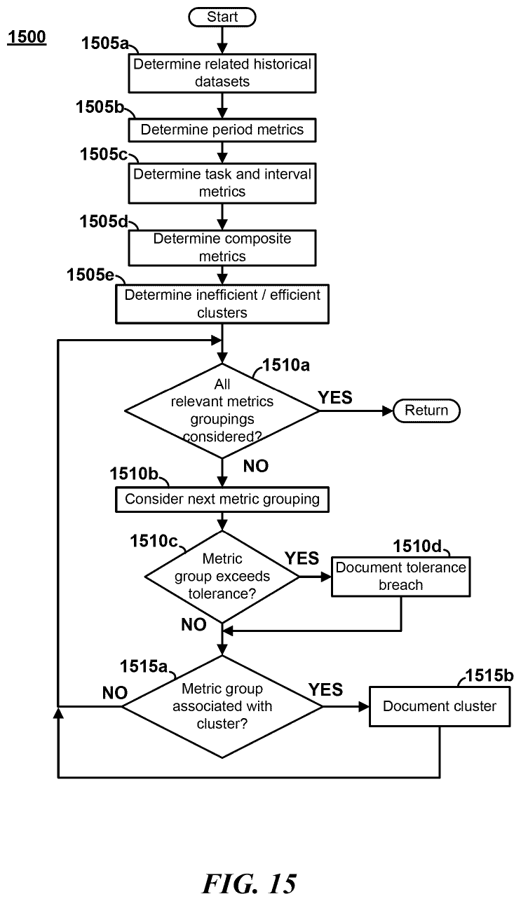

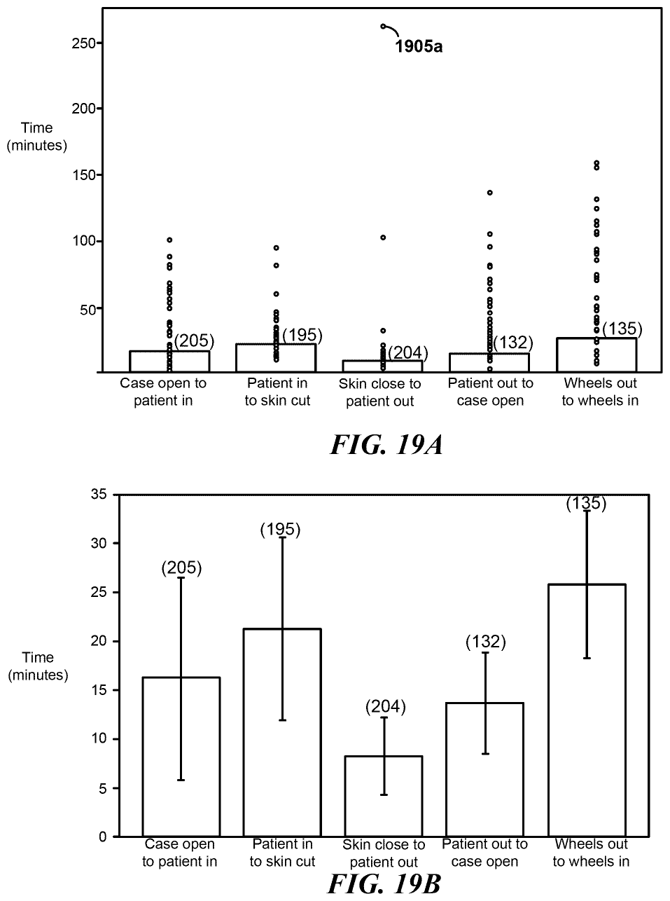

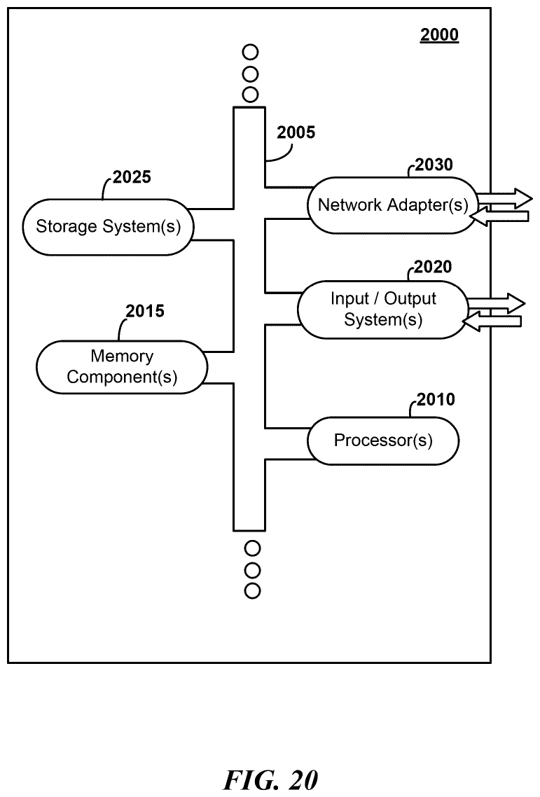

Specifically, FIG. 8 is a schematic block diagram illustrating various metrics and their relations in constructing an “ORA score” as may be performed in some embodiments. Within the temporal grouping 805, an “efficiency” scoring metric 805a may combine the nonoperative metrics that measure temporal workflow efficiency in an OR, e.g., the duration of one or more of the six temporal interval metrics of FIG. 5A. More specifically, the nonoperative metrics, averaged, as a mean or median, over all cases collected from a team, theater, or hospital, may be compared to the top 20% teams, theaters, or hospitals (e.g., as manually indicated by reviewers or from historical patterns via iterations of topology 450) in a database as a benchmark. A “consistency” metric 805b may combine (e.g., sum or find the mean or median) the standard deviations of nonoperative metrics (e.g., the six temporal interval metrics of FIG. 5A) across all cases collected from a current team, theater, or hospital. An “adverse event” metric 805c may combine (e.g., sum) negative outliers, e.g., as detected in terms of the interval metrics of FIGS. 5A-B. Outliers may, e.g., be detected using statistical analysis algorithms (e.g., clustering, distribution analysis, regression, etc. as discussed herein with reference to FIGS. 15, 16, 19A-B, and 20A-B). Negative outliers may be identified as those for which at least one of the nonoperative interval metrics of FIGS. 5A-B metrics are outside a threshold, such as a standard deviation, from than the relevant team, theater, or hospital median or mean (e.g., based on a threshold specified by an expert reviewer or upon historical patterns from past iterations of topology 450). Examples of such outliers are discussed herein, e.g., with respect to FIGS. 19A-B and FIGS. 20A-B.

Within the scheduling grouping 810, a “case volume” scoring metric 810a includes the mean or median number of cases operated per OR, per day, for a team, theater, or hospital, normalized by the expected case volume for a typical OR (e.g., again, as designated in a historical dataset benchmark, such as a mean or median). A “first case turnovers” scoring metric 810b is the ratio of first cases in an operating day that were turned over compared to the total number of first cases captured from a team, theater, or hospital. Alternatively, a more general “case turnovers” metric is the ratio of all cases that were turned-over compared to the total number of cases as performed by a team, in a theater, or in hospital. A “delay” scoring metric 810c is a mean or median positive (behind a scheduled start time of an action) or negative (before a scheduled start time of an action) departure from a scheduled time in minutes for each case, normalized by the acceptable delay (e.g., a historical mean or median benchmark). Naturally, the negative or positive definition may be reversed (e.g., wherein starting late is instead negative and starting early is instead positive) if other contextual parameters are likewise adjusted.

Within the human resource metrics grouping 815, a “headcount to complete tasks” scoring metric 815a combines the mean or median headcount (the largest number of detected personnel throughout the procedure in the OR at one time) over all cases collected for the team, theater, or hospital needed to complete each of the temporal nonoperative tasks for each case, normalized by the recommended headcount for each task (e.g., a historical benchmark median or mean). An “OR Traffic” scoring metric 815b measures the mean amount of motion in the OR during each case, averaged (itself as a median or mean) over all cases collected for the team, theater, or hospital, normalized by the recommended amount of traffic (e.g., based upon a historical benchmark as described above). For example, this metric may receive (two or three-dimensional) optical flow, and convert such raw data to a single numerical value, e.g., an entropy representation, a mean magnitude, a median magnitude, etc.

Within the “other” metrics grouping 815, a “room layout” scoring metric 820a includes a ratio of robotic cases with multi-part roll-ups or roll-backs, normalized by the total number of robotic cases for the team, theater, or hospital. That is, ideally, each roll up or back of the robotic system would include a single motion. When, instead, the team member moves the robotic system back and forth, such a “multi-part” roll implies an inefficiency, and so the number of such multi-part rolls relative to all the roll up and roll back events may provide an indication of the proportion of inefficient attempts. As indicated by this example, some metrics may be unique to robotic theaters, just as some metrics may be unique to nonrobotic theaters. In some embodiments, correspondences between metrics unique to each theater-type may be specified to facilitate their comparison. A “modality conversion” scoring metric 820b includes a ratio of cases that have both robotic and non-robotic modalities normalized by the total number of cases for the team, theater, or hospital. For example, this metric may count the number of conversions, e.g., transitioning from a planned robotic configuration to a nonrobotic configuration, and vice versa, and then dividing the total number of such cases with such a conversion by the total cases. Whether occurring in an operative or nonoperative periods, such conversions may be reflective of inefficiencies in nonoperative periods (e.g., improper actions in a prior nonoperative period may have rendered the planned robotic procedure in the operative period impractical). Thus, this metric may capture inefficiencies in planning, in equipment, or in unexpected complications in the original surgical plan.

While each of the metrics 805a-c, 810a-c, 815a-c, and 820a-b may be considered individually to assess nonoperative period performances, or in combinations of the multiple of the metrics, as discussed above with respect to EQN. 1, some embodiments consider an “ORA score” 830 reflecting an integrated 825 representation of all these metrics. When, e.g., presented in combination with data of the duration of one or more of the intervals in FIG. 5A-C, the ORA score may provide a readily discernible means for reviewers to quickly and intuitively assess the relative performance of surgical teams, surgical theaters, hospitals and hospital systems, etc. during nonoperative periods, across theaters, across teams, across types of surgical procedures (nonoperative periods before or after prostatectomies, hernia repair, etc.), types of surgical modalities (nonoperative periods preparing for, or resetting after, nonrobotic laparoscopic procedures, nonrobotic open procedures, robotic procedures, etc.), hospital systems, etc.

Accordingly, while some embodiments may employ more complicated relationships (e.g., employing any suitable mathematical functions and operations) between the metrics 805a-c, 810a-c, 815a-c, and 820a-b in forming the ORA score 830, in this example, each of the metrics may be weighted by a corresponding weighting value 850a-j such that the integrating 825 is a weighted sum of each of the metrics. The weights may be selected, e.g., by a hospital administrator or reviewers in accordance with which of the metrics are discerned to be more vital to current needs for efficiency improvement. For example, in a system where reviewers wish to assess whether reports that limited staff are affecting efficiency, then the weight 850g may be upscaled relative to the other weights. Thus, when the ORA score 830 across procedures is compared in connection with the durations of one or more of the intervals in FIG. 5A-C for the groups of surgeries, the reviewer can more readily discern if there exists a relation between the head count and undesirable interval durations. Naturally, one will appreciate other choices and combinations of weight adjustment, as well as particular consideration of specific interval durations, to assess other performance characteristics.

### Example Metric Scoring Methodologies—ORA Significance Assessment

Some higher ORA composite metrics scores may positively correlate with increased system utilization u and reduced OR minutes per case t for the hospitals in a database, e.g., as represented by EQN. 2:

$${p}_{s , u} = \frac{cov ( s , u )}{{σ}_{s} {σ}_{u}} \geq 0.75 \qquad ( 2 )$$

Thus, the ORA composite score may be used for a variety of analysis and feedback applications. For example, the ORA composite score may be used to detect negative trends and prioritize hospitals, theaters, teams, or team members, that need workflow optimizations. The ORA composite score may also be used to monitor workflow optimizations, e.g., to verify adherence to requested adjustments, as well as to verify that the desired improvements are, in fact, occurring. The ORA composite score may also be used to provide an objective measure of efficiency for when teams perform new types of surgeries for the first time.

### Example Metric Scoring Methodologies—Additional Metrics

Additional metrics to assess workflow efficiency may be generated by compositing time, staff count, and motion metrics. For example, a composite score may consider scheduling efficiency (e.g., a composite formed from one or more of case volume 810a, first case turnovers 810b, and case delay 810c) and one or both of modality conversion 820b and the duration of an “idle time” metric, which is a mean or median of the idle time (for individual members or teams collectively) over a period (e.g., during action 525b).

Though, for convenience, sometimes described as considering the behavior of one or more team members, one will appreciate that the metrics described herein may be used to compare the performances of individual members, teams, theaters (across varying teams and modalities), hospitals, hospital systems, etc. Similarly, metrics calculated at the individual, team, or hospital level may be aggregated for assessments of a higher level. For example, to compare hospital systems, metrics for team members within each of the systems, across the system's hospitals, may be determined, and then averaged (e.g., a mean, median, sum weighted by characteristics of the team members, etc.) for a system-to-system comparison.

### Example Nonoperative Data Processing Workflow

FIG. 9A is a schematic block diagram depicting a general processing flow as may be implemented in some embodiments. Specifically, this example flow employs various machine learning consolidation systems for producing elemental OR metrics (such as temporal interval durations, personnel presence, personnel motion, equipment motion, etc., from which other metrics, e.g., as described in FIG. 8, may be generated) from the raw multimodal theater-wide sensor data.

In some embodiments (e.g., where the data has not been pre-processed), a nonoperative segment detection module 905a may be used to detect nonoperative segments from full-day theater-wide data. A personnel count detection module 905b may then be used to detect a number of people involved in each of the detected nonoperative segments/activities of the theater-wide data (e.g., a spatial-temporal machine learning algorithm employing a 3D convolutional network for handing visual image and depth data over time, e.g., as appearing in video). A motion assessment module 905c may then be used to measure the amount of motion (e.g., people, equipment, etc.) observed in each of the nonoperative segment/activities (e.g., using optical flow methods, a machine learning tracking system, etc.). A metrics generation component 905d may then be used to generate metrics, e.g., as disclosed herein (e.g., determining as metrics the temporal durations of each of the intervals and actions of FIGS. 5A-C and the metrics as discussed in FIG. 8). While metrics results may be presented directly to the reviewer in some embodiments, as described herein, some embodiments may instead provide some initial analytical assessment of the metric values, determining standard deviations relative to historical values, prioritizing greater tolerance departures for prioritized presentation to the reviewer, determining if metric values (e.g., motion) indicate that it would be desirable to perform a more refined analysis of the data (e.g., determining team member movement paths, object collision event detections, etc.), etc. Accordingly, a metrics analysis component 905e may then analyze the generated metrics, e.g., to determine outliers relative to historical patterns.

FIG. 9B is a schematic block diagram depicting elements in a more detailed example processing flow than the flow depicted in FIG. 9A, as may be implemented in some embodiments. One will appreciate that each depicted component may be logic or may be one or more machine learning systems, as discussed in greater detail herein. The computer system 910b may receive the theater wide sensor data 910a and first perform the nonoperative period detection 910c (e.g., identifying the periods 310a, 310b, 310c, 310d, though some systems may be configured to only detect nonoperative periods of the character of periods 310b, and 310c). Once the portions of the theater-wide data corresponding to the nonoperative periods have been detected, the data may then be further segmented into corresponding action tasks or intervals (e.g., the intervals 550a-d and/or groupings 510, 515, 520, 525 and respective action tasks) at block 910d.

Using object detection (and in some embodiments, tracking) machine learning systems 910e, the system may perform object detection using machine learning methods, such as of equipment 910f or personnel 910h (ellipsis 910g indicating the possibility of other machine learning systems). In some embodiments, only personnel detection 910h is performed, as only the number of personnel and their motion are needed for the desired metrics. Motion detection component 910i may then analyze the objects detected at block 910e to determine their respective motions, e.g., using various machine learning methods, optical flow, combinations thereof, etc. disclosed herein.

Using the number of objects, detected motion, and determined interval durations, a metric generation system 910j may generate metrics (e.g., the interval durations may themselves serve as metrics, the values of FIG. 8 may be calculated, etc.). The metric values may then be analyzed via component 910k to determine, e.g., outliers and other deviations from historical data (e.g., previous iterations of the topology 450). The system may consider 915a, 915c historical sensor data 915e and historical metrics data 915f when performing the historical comparison at block 910k (e.g., clustering historical metric values around efficient and inefficient nodes, then assessing the newly arrived data's distance to these nodes). In this manner, the system may infer that entire teams, groups of members, or individual members performed subpar compared to historical metrics data for similar roles, team member compositions, or individual team members. Conversely, the processed and raw theater-wide sensor data may be provided 915b to the historical data storage 915e for use in future analysis. Similarly, the metrics results and outlier determinations may be recorded 915d in the historical metrics database 915f for future reference.

The results of the analysis may then be presented via component 9101 (e.g., sent over a network to one or more of applications 550f) for presentation to the reviewer. For example, application algorithms may consume the determined metrics and nonoperative data and propose customized actionable coaching for each individual in the team, as well as the team as a whole, based upon metrics analysis results (though such coaching or feedback may first be determined on the computer system 910b in some embodiments). Example recommendations include, e.g.: changes in the OR layout at various points in time, changes in OR scheduling, changes in communication systems between team members, changes in numbers of staff involved in various tasks, etc. In some embodiments, such coaching and feedback may be generated by comparing the metric values to a finite corpus of known inefficient patterns (or conversely, known efficient patterns) and corresponding remediations to be proposed (e.g., slow port placement and excess headcount may be correlated with an inefficiency resolved by reducing head count for that task).

For further clarity, FIG. 9C is a flow diagram illustrating various operations in an example overall process 920 for analyzing theater-wide data. At block 920a, the computer system may receive the theater-wide sensor data for the theater to be examined. At block 920b, the system may perform pre-processing on the data, e.g., reconciling theaterwide data to a common format, as when fisheye and rectilinear sensor data are both to be processed.

At block 920c, the system may perform operative and nonoperative period recognitions, e.g., identifying each of the segments 310a-d and 315a-c from the raw theater wide sensor data. In some embodiments, such divisions may be recognized, or verified, via ancillary data, e.g., console data, instrument kinematics data, etc. (which may, e.g., be active only during operative periods).

The system may then iterate over the detected nonoperative periods (e.g., periods 310a, 310b) at blocks 920d and 925a. In some embodiments, operative periods may also be included in the iteration, e.g., to determine metric values that may inform the analysis of the nonoperative segments, though many embodiments will consider only the nonoperative periods. For each period, the system may identify the relevant tasks and intervals at block 925b, e.g., the intervals, groups, and actions of FIGS. 5A-C.

At blocks 925c and 925e, the system may iterate over the corresponding portions of the theater data for the respectively identified tasks and intervals, performing object detections at block 925f, motion detection at block 925g, and corresponding metrics generation at block 925h. In some embodiments, at block 925f, only a number of personnel in the theater may be determined, without determining their roles or identities. Again, the metrics may thus be generated at the action task level, as well as at the other intervals described in FIGS. 5A-C. In alternative embodiments, the metrics may simply be determined for the nonoperative period (e.g., where the duration of the intervals 550a-e are the only metrics to be determined).

After all the relevant tasks and intervals have been considered for the current period at block 925c, then the system may create any additional metric values (e.g., metrics including the values determined at block 925h across multiple tasks as their component values) at block 925d. Once all the periods have been considered at block 920d the system may perform holistic metrics generation at block 930a (e.g., metrics whose component values depend upon the period metrics of block 925d and block 925h, such as certain composite metrics described herein).

At block 930b, the system may analyze the metrics generated at blocks 930a, 925d, and at block 925h. As discussed, many metrics (possibly at each of blocks 930a, 925h, and 925d) will consider historical values, e.g., to normalize the specific values here, in their generation. Similarly, at block 930b the system may determine outliers as described in greater detail herein, by considering the metrics results in connection with historical values. Finally, at block 930c, the system may publish its analysis for use, e.g., in applications 450f.

### Example Nonoperative Theater-Wide Data Processing—Nonoperative Data Recognition

One will appreciate a number of systems and methods sufficient for performing the operative/nonoperative period detection of components 905a or 910c and activity/task/interval segmentation of block 910d (e.g., identifying the actions, tasks, or intervals of FIGS. 5A-C). Indeed, as mentioned, in some embodiments, alternative signals than the theater-wide data or monitoring of gross-signals in the theater-wide data may suffice for distinguishing periods 310a-d from periods 315a-d. For example, in some embodiments, a team member may provide explicit notification. Similarly, the absence of kinematics and system events data from robotic surgical systemics consoles or instruments may indicate a prolonged separation between the surgeon and patient or between a robotic platform and the patient, which may suffice to indicate that an inter-surgical nonoperative period has begun (or provide verification of a machine learning system's parallel determination).

However, some embodiments consider instead, or in addition, employing machine learning systems for performing the nonoperative period detection. For example, some embodiments employ spatiotemporal model architectures, e.g., like a transformer architecture such as that described in Bertasius, Gedas, Heng Wang, and Lorenzo Torresani. “Is Space-Time Attention All You Need for Video Understanding?” arXiv™ preprint arXiv™:2102.05095 (2021). Such approaches may also be especially useful for automatic activity detection from long sequences of theater-wide sensor data. The spatial segment transformer architecture may be designed to learn features from frames of theater-wide data (e.g., visual image video data, depth frame video data, visual image and depth frame video data, etc.). The temporal segment may be based upon a gated recurrent unit (GRU) method and designed to learn the sequence of actions in a long video and may, e.g., be trained in a fully supervised manner (again, where data labelling may be assisted by the activation of surgical instrument data). For example, OR theater-wide data may be first annotated by a human expert to create ground truth labels and then fed to the model for supervised training.

Some embodiments may employ a two-stage model training strategy: first training the back-bone transformer model to extract features and then training the temporal model to learn a sequence. Input to the model training may be long sequences of theater-wide data (e.g., many hours of visual image video) with output time-stamps for each segment (e.g., the nonoperative segments) or activity (e.g., intervals and tasks of FIGS. 5A-C) of interest. One will appreciate that some models may operate on individual visual images, individual depth frames, groups of image frames (e.g., segments of video), groups of depth frames (e.g., segments of depth frame video), combinations of visual video and depth video, etc.

As another example, FIG. 10 is a flow diagram illustrating various operations in an example process 1005 for performing nonoperative period detection in some embodiments. Specifically, as the number of theater-wide sensors may change across theaters, or across time in the same theater, it may be undesirable to invest in training a machine learning system configured to receive only a specific number of theater-wide data inputs. Thus, in these embodiments, where the classifier is not configured to consider the theater-wide sensor data from all the available streams at once, the system may instead consider the streams individually, or in smaller groups, and then analyze the collective results, e.g., in combination with smoothing operations, so as to assign a categorization to the segment under consideration.

For example, after receiving the theater-wide data at block 1005a (e.g., all of three streams 325a-e, 330a-e, and 335a-e) the system may iterate over the data in intervals at blocks 1005b and 1005c. For example, the system may consider the streams in successive segments (e.g., 30 second, one, or two minute intervals), though the data therein may be down sampled depending upon the framerate of its acquisition. For each interval of data, the system may iterate over the portion of the interval data associated with the respective sensor's streams at blocks 1010a and 1010b (e.g., each of streams 325a-e, 330a-e, and 335a-e or groups thereof, possibly considering the same stream more than once in different groupings). For each stream, the system may determine the classification results at block 1010c as pertaining to an operative or nonoperative interval. After all the streams have been considered, at block 1010d, the system may consider the final classification of the interval. For example, the system may take a majority vote of the individual stream classifications of block 1010c, resolving ties and smoothing the results based upon continuity with previous (and possibly subsequently determined) classifications.

After all the theater-wide data has been considered at block 1005b, then at block 1015a the system may consolidate the classification results (e.g., performing smoothing and continuity harmonization for all the data, analogous to that discussed with respect to block 1010d, but here for larger smoothing windows, e.g., one to two hours). At block 1015b, the system may perform any supplemental data verification before publishing the results. For example, if supplemental data indicates time intervals with known classifications, the classification assignments may be hardcoded for these true positives and the smoothing rerun.

### Example Nonoperative Theater-Wide Data Processing—Object Recognition

Like nonoperative and operative theater-wide data segmentation, one will likewise appreciate a number of ways for performing object detection (e.g., at block 905b or component 910e). Again, in some embodiments, object detection includes merely a number of personnel count, and so a You Only Look Once (YOLO) style network (e.g., as described in Redmon, Joseph, et al. “You Only Look Once: Unified, Realtime Object Detection.” arXiv™ preprint arXiv™:1506.02640 (2015)), perhaps applied iteratively, may suffice. However, some embodiments consider using groups of visual images or depth frames. For example, some embodiments employ a transformer based spatial model to process frames of the theater-wide data, detecting all humans present and reporting the number. An example of such architecture is described in Carion, Nicolas, et al. “End-to-End Object Detection with Transformers.” arXiv™ preprint arXiv™:2005.12872 (2020).

To clarify this specific approach, FIG. 11A is a schematic block diagram illustrating an example information processing flow as may be used for performing object detection in connection with some embodiments. Given a visual or depth frame image 1105f, the system may present the image's raw pixel or depth values to a convolutional network 1105a trained to produce image features 1105b. These features may in turn be provided to a transformer encoder-decoder 1105c and the bipartite matching loss 1105d used to make predictions 1105e for the location and number of objects (e.g., personnel or equipment) in the image, reflected here by bounding boxes within the augmented image 1105g (one will appreciate that an actual augmented image may not be produced by the system, but rather, only indications of the object locations and, in some embodiments, of the type of object found therein).

FIG. 11B is a flow diagram illustrating various operations in an example process 1100 for performing object detection as may be used in connection with some embodiments. At block 1110a, the system may receive the theater-wide data (visual image data, depth data, etc.). At blocks 1110b, and 1110c, as in the process 1005, the system may iterate over the nonoperative periods, considering the data in discrete, successive intervals (as mentioned, in some embodiments the operative periods may be considered as well, e.g., to verify continuity with the object detections and recognitions at the beginnings or ends of the nonoperative periods).

At blocks 1110d and 1115a the system may consider groups of theater-wide data. For example, some embodiments may consider every moment of data capture, whereas other embodiments may consider every other capture or captures at intervals, since some theater sensors may employ high data acquisition rates (indeed, not all sensors in the theater may apply a same rate and so normalization may be applied so as to consolidate the data). For such high rates, it may not be reasonable to interpolate object locations between data captures if the data capture rate is sufficiently larger than the movement speeds of objects in the theater. Similarly, some theater sensor's data captures may not be perfectly synchronized, or may capture data at different rates, obligating the system to interpolate or to select data captures sufficiently corresponding in time so as to perform detection and metrics calculations.

At blocks 1115b and 1115c, the system may consider the data in the separate theater-wide sensor data streams and perform object detection at block 1115d, e.g., as described above with respect to FIG. 11A, or using a YOLO network, etc. After object detection has been performed for each stream for the group under consideration, the system may perform post-processing at block 1115e. For example, if the relative poses of the theater-wide sensors are known within the theater, then their respective object detections may be reconciled to better confirm the location of the object in a three-dimensional representation such as a three-dimensional point cloud. Similarly, the relative data captures may be used to verify one another's determinations and to resolve occlusions based upon temporal continuity (e.g., as when a team member occludes one senor's perspective, but not another sensor's).

After all of the temporal groups have been considered at block 1110d, then at block 1110e, additional verification may be performed, e.g., using temporal information from across the intervals of block 1110d to reconcile occlusions and lacuna in the object detections of block 1115d. Once all the nonoperative periods of interest have been considered at block 1110b, at block 1120a, the system may perform holistic post-processing and verification in-filling. For example, knowledge regarding object presence between periods or based upon a type of theater or operation may inform the expected numbers and relative locations of objects to be recognized. To this end, even though some embodiments may be interested in analyzing nonoperative periods exclusively, the beginning and end of operative periods may help inform or verify the nonoperative period object detections, and may be considered. For example, if four personnel are consistently recognized throughout an operative period, then the system should expect to identify four personnel at the end of the preceding, and the beginning of the succeeding, nonoperative periods.

### Example Nonoperative Theater-Wide Data Processing—Object Tracking

As with segmentation of the raw data into nonoperative periods (e.g., as performed by nonoperative period detection component 910c), and the detection of objects, such as personnel, within those periods (e.g., via component 910e), one will appreciate a number of ways to perform tracking and motion detection. For example, object detection, as described, e.g., in FIG. 11B, in combination with optical flow analysis (with complementary stream perspectives resolving ambiguities) may readily be used to recognize each particular object's movement throughout the theater. As another example, some embodiments may employ multi-object machine learning tracking algorithms, which involve detecting and tracking multiple objects within a sequence of theater-wide data. These approaches may identify and locate objects of interest in each frame and then associate those objects across frames to keep track of their movements over time. For example, some embodiments may use an implementation analogous to that described in Meinhardt, Tim, et al. “TrackFormer: Multi-Object Tracking with Transformers.” arXiv™ preprint arXiv™:2101.02702 (2021).

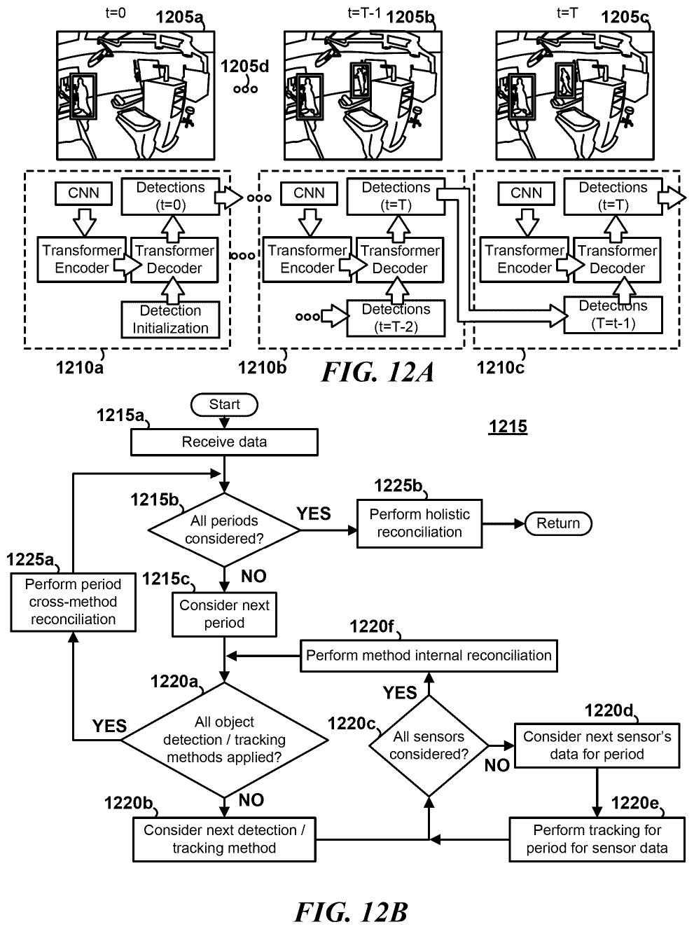

As an example in accordance with the approach of Meinhardt, et al., FIG. 12A is schematic block diagram illustrating an example tracking information processing flow as may be used in connection with some embodiments. In a first visual image or depth frame 1205a, the system may apply a tracking framework collection 1210a of convolution neural network, transformer encoders and decoders, and initial object detection (e.g., with the assistance of the object detection method of FIG. 11A). Iterative application 1210b and 1210c of the tracking framework to subsequent images or frames 1205b and 1205c may produce object detections, such as personnel, with a record of the positions across the frames 1205a, 1205b, 1205c (ellipsis 1205d reflect the presence of intervening frames and tracking recognitions).

FIG. 12B is flow diagram illustrating various operations in an example process 1215 for performing object tracking as may be used in connection with some embodiments. At block 1215a, the system may receive the theater-wide data, e.g., following nonoperative period identification. At blocks 1215b and 1215c the system may iterate over the nonoperative periods and for each period, iterate over the contemplated detection and tracking methods at blocks 1220a and 1220b. For each method, the sensor data streams may be considered in turn at blocks 1220c and 1220d, performing the applicable detection and tracking method at block 1220e (one will appreciate that alternatively, in some embodiments, the streams may be first integrated before applying the object detection and tracking systems, as when simultaneously acquired depth frames from multiple sensors are consolidated into a single virtual model). As mentioned, some methods may benefit from considering temporal and spatial continuity across the theater-wide sensors, and so reconciliation methods for the particular tracking application may be applied at block 1220f.

Similarly, reconciliation between the tracking methods' findings across the period may be performed at block 1225a. For example, determined locations for objects found by the various methods may be averaged. Similarly, the number of objects may be determined by taking a majority vote among the methods, possibly weighted by uncertainty or confidence values associated with the methods. Similarly, after all the nonoperative periods have been considered, the system may perform holistic reconciliation at block 1225b, e.g., ensuring that the initial and final object counts and locations agree with those of neighboring periods or action groups.

As one will note when comparing FIG. 12B and FIG. 9C, object detection, tracking, or motion detection may be performed at the period level (and then associated with tasks/actions/intervals for metrics calculation if desired) or may be performed after the actions, tasks, or intervals have been identified, and upon corresponding data specifically.

### Example Nonoperative Theater-Wide Data Processing—Motion Assessment

While some tracking systems may readily facilitate motion analysis at motion detection component 910i, some embodiments may alternatively, or in parallel, perform motion detection and analysis using visual image and depth frame data. In some embodiments, simply the amount of motion (in magnitude, regardless of its direction component) within the theater in three-dimensional space of any objects, or of only objects of interest, may be useful for determining meaningful metrics during nonoperative periods. However, more refined motion analysis may facilitate more refined inquiries, such as team member path analysis, collision detection, etc.

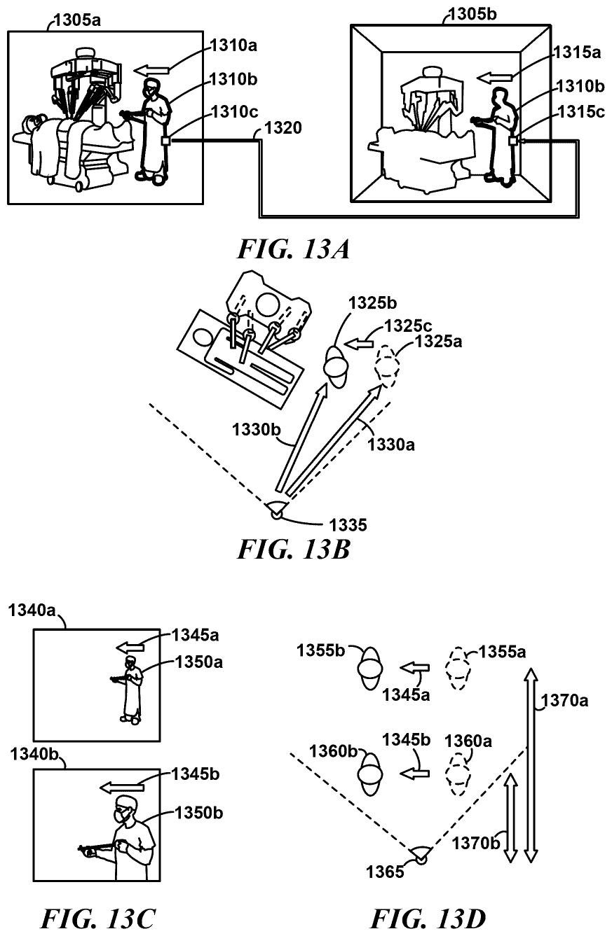

As an example optical-flow based motion assessment, FIG. 13A is a schematic visual image 1305a and depth frame 1305b theater-wide data pair, with an indication of the optical-flow derived correspondence as may be used in some embodiments. Specifically, the data processing system may review sequences of visual image data to detect optical flow. Here, the system has detected that the team member 1310b is moving from the right to the left of the image as indicated by arrow 1310a and by the pixel border around the pixels having optical flow around team member 1310b.

While some embodiments may consider motion based upon the optical flow from visual images alone, it may sometimes be desirable to “standardize” the motion. Specifically, turning to FIG. 13C, movement 1345a far from the camera, as shown in image 1340a may result in a smaller number of pixels (the pixels depicting the member 1350a) being associated with the optical flow. Conversely, as shown in image 1340b, when the team member 1350b is very close to the sensor, their motion 1345b may result in an optical flow affecting many more pixels.

Rather than allow the number of visual image pixels involved in the flow to affect the motion determination, some embodiments may standardize the motion associated with the optical flow to three-dimensional space. That is, with reference to FIG. 13D, the motions 1345a and 1345b may be the same in magnitude in three-dimensional space, as the team members move from locations 1355a, 1360a to locations 1355b, 1360b, respectively. While the locations 1360a-b are a smaller distance 1370b from the sensor 1365 than the distance 1370a from the sensor 1365 to the locations 1355a-b, some embodiments may seek to identify the same amount of motion 1345a, 1345b in each instance. Specifically, downstream metrics may treat the speed of the motions 1345a, 1345b equally, regardless of their distance from the capturing sensor.

To accomplish this, returning to FIG. 13A, for each portion of the visual image 1305a associated with the optical flow, the system may consider the corresponding portions of the simultaneously acquired depth image 1305b, here, where the team member 1310b and their motion, indicated by arrow 1315a, will also be manifest. That is, in this example the pixels 1310c associated with the optical flow may correspond 1320 to the depth values 1315c. By considering these depth values 1320, the system may infer the distance to the object precipitating the optical flow (e.g., one of distances 1370b and 1370a). That is, with reference to FIG. 13B, the system may be able to infer the “standardized” motion 1325c in three-dimensional space for the object moving from position 1325a to position 1325b, once the distances 1330a and 1330b from the capturing sensor 1335 have been inferred from the depth data. In some embodiments, in lieu of first detecting optical flow in the visual image, optical flow in the depth data may instead be used and the standardized motion determined mutatis mutandis.

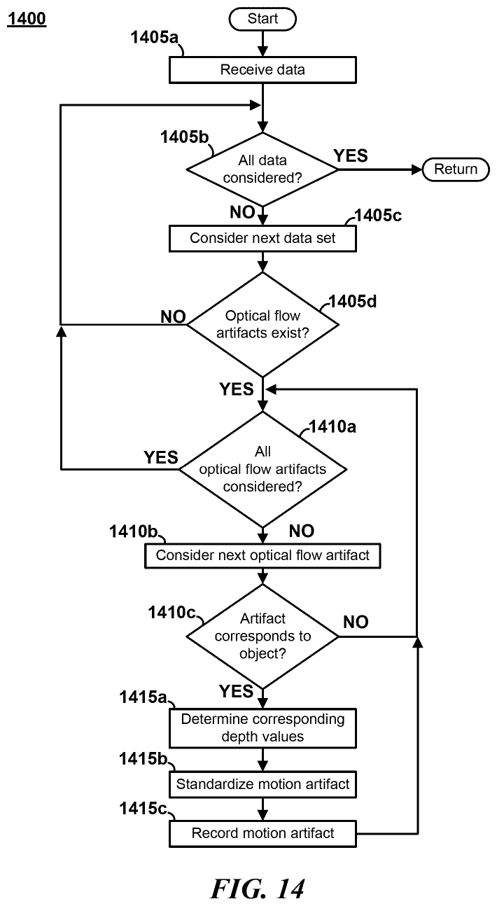

FIG. 14 is a flow diagram illustrating various operations in an example process 1400 for performing motion analysis from theater-wide data, as may be applied in some embodiments. At blocks 1405b and 1405c, the system may iterate over the theater-wide data received at block 1405a. For example, theater-wide data may be down sampled and considered in discrete data sets of temporally successive visual image and depth frame pairs. Where one or more optical flow artifacts (contiguous regions with optical flow above a threshold are detected in either the visual images or the depth frames) are detected within the data set at block 1405d, the system may iterate over the artifacts at blocks 1410a and 1410b. Many artifacts may not correspond to objects of interest for preparing metrics. For example, incidental motion of some equipment, adjustment of some lights, opening of some doors, etc., may not be relevant to the downstream analysis. Accordingly, at block 1410c, the system may verify that the artifact is associated with one or more of the objects of interest (e.g., the personnel or equipment detected using the methods disclosed herein via the machine learning systems of component 910e, e.g., including the systems and methods of FIGS. 11A-B and 12A-B). for example, pixels corresponding to the optical flow may be compared with pixels identified in, e.g., a YOLO network object detection. In some cases, a single optical flow artifact may be associated with more than one object, e.g., when one moving object occludes another moving object. Assessment of the corresponding depth values may reveal the identities of the respective objects appearing in the artifact or at least their respective locations and trajectories.

Thus, where the artifact corresponds to an object of interest (e.g., team personnel), then at block 1415a, the system may determine the corresponding depth values and may standardize the detected motion at block 1415b to be in three-dimensional space (e.g., the same motion value regardless of the distance from the sensor) rather than in the two-dimensional plane of a visual image optical flow, e.g., using the techniques discussed herein with respect to FIGS. 13A-D. The resulting motion may then be recorded at block 1415c for use in subsequent metrics calculation as discussed in greater detail herein.

### Example Nonoperative Theater-Wide Metrics Analysis—Outlier Detection

Following metrics generation (e.g., at metric generation system 910j) some embodiments may seek to recognize outlier behavior (e.g., at metric analysis system 910k) to detect outliers in each team/operating room/hospital/etc. based upon the above metrics, including the durations of the actions and intervals in FIGS. 5A-C, the numbers of people involved in each theater and the amount of motion observed, etc. For example, FIG. 15 is flow diagram illustrating various operations in an example process 1500 for outlier analysis based upon the determined metric values, as may be implemented in some embodiments.

At block 1505a, the system may acquire historical datasets, e.g., for use with metrics having component values (such as normalizations) based upon historical data. At block 1505b, the system may determine metrics results for nonoperative period as a whole (e.g., cumulative motion within the period, regardless of whether it occurred in association with any particular task or interval). At block 1505c, the system may determine metrics results for specific tasks and intervals within each of the nonoperative segments (e.g., the durations of actions and intervals in FIGS. 5A-C). At block 1505d, the system may then determine composite metric values from the previous of the determined metrics (e.g., the ORA score 830 discussed in FIG. 8).

At block 1505e, clusters of metric values corresponding to patterns of inefficient or efficient nonoperative theater states, as well as clusters of metric values corresponding to patterns of efficient or positive nonoperative theater states, may be included in the historical data of block 1505a. Such clusters may be used both to find metric scores, and patterns of metrics scores, distance from ideal clusters and distance from undesirable clusters (e.g., where the distance is the Euclidean distance and each metric of a group is considered as a separate dimension).

Thus, the system may the iterate over the metrics individually, or in groups, at blocks 1510a and 1510b to determine if the metrics or groups exceed a tolerance at block 1510c relative to the historical data clusters (naturally, the nature of the tolerance may change with each expected grouping and may be based upon a historical benchmark, such as one or more standard deviations from a median or mean). Where such tolerance is exceeded (e.g., metric values or groups of metric values are either too close to inefficient clusters or too far from efficient clusters), the system may document the departure at block 1510d for future use in coaching and feedback as described herein.

For clarity, as mentioned, the cluster may occur in an N dimensional space where there are N respective metrics considered in the group (though alternative spaces and surfaces for comparing metric values may also be used). Such an algorithm may be applied to detect outliers for each team/operating room/hospital based upon the above metrics. Cluster algorithms (e.g., based upon K-means, using machine learning classifiers, etc.) may both reveal groupings and identify outliers, the former for recognizing common inefficient/efficient patterns in the values, and the latter for recognizing, e.g., departures from ideal performances or acceptable avoidance of undesirable states.

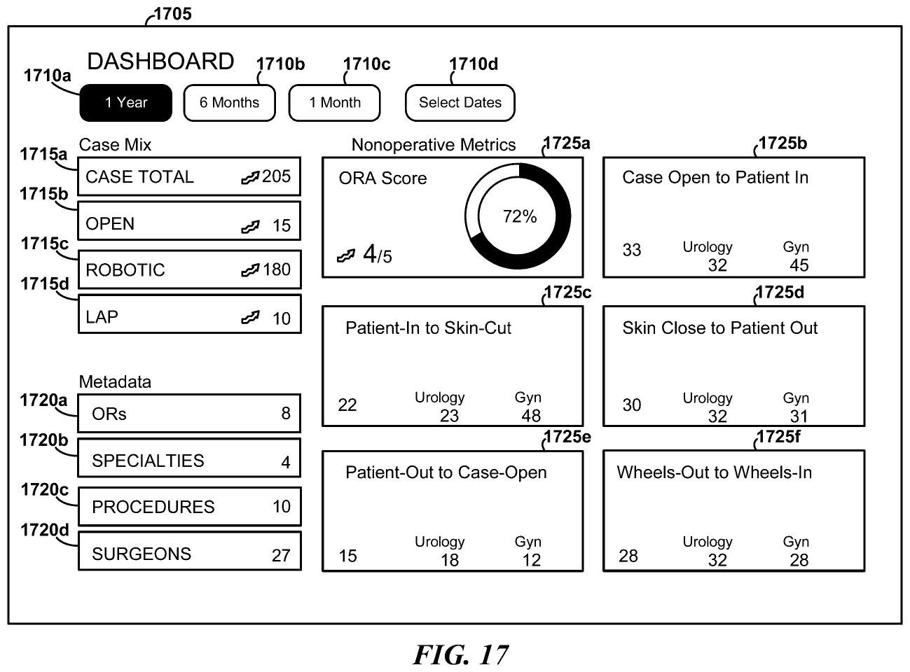

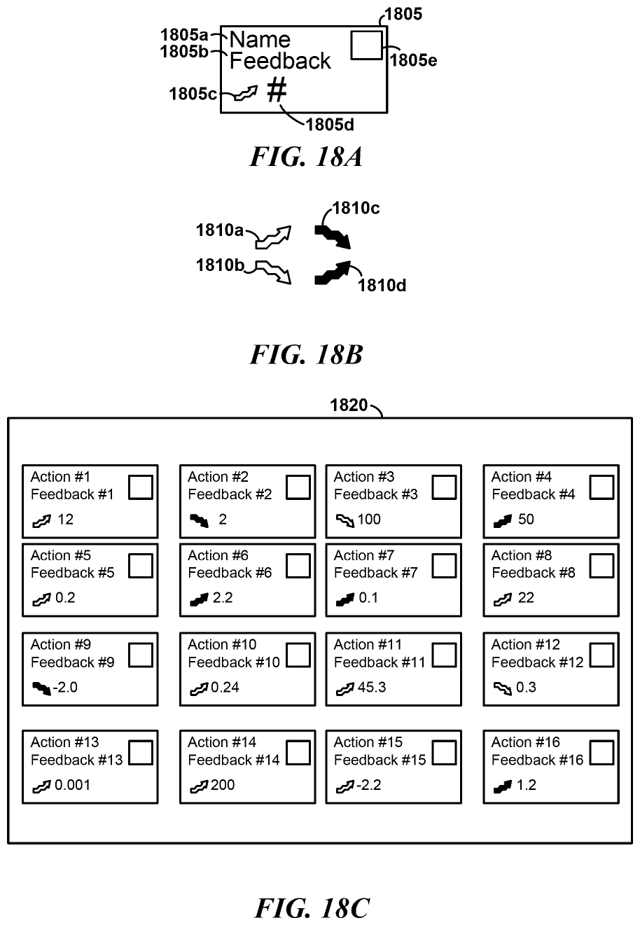

Thus the system may determine whether the metrics individually, or in groups, are associated (e.g., within a threshold distance of, such as the cluster's standard deviation, larges principal component, etc.) with an inefficient, or efficient, cluster at block 1515a, and if so, document the cluster for future coaching and feedback at block 1515b. For example, raw metric values, composite metric values, outliers, distances to or from clusters, correlated remediations, etc., may be presented in a GUI interface, e.g., as will be described herein with respect to FIG. 17 or 18A-C.

### Example Nonoperative Data Analysis—Coaching

Following outlier detection and clustering, in some embodiments, the system may also seek to consolidate the results into a form suitable for use by feedback and coaching (e.g., by the applications 550f). For example, remediating actions may already be known for tolerance breaches (e.g., at block 1510c) or nearness to adverse metrics clusters (e.g., at block 1515a). Here, coaching may, e.g., simply include the known remediation when reporting the breach or clustering association.

Some embodiments may recognize higher level associations in the metric values, from which remediations may be proposed. For example, after considering a new dataset from a theater in a previously unconsidered hospital, various embodiments may determine that a specific surgical specialty (e.g., Urology) in that theater, possesses a large standard deviation in its nonoperative time metrics. Various algorithms disclosed herein may consume such large standard deviations, other data points, and historical data and suggest corrective action regarding with scheduling or staffing model. For example, a regression model may be used that employs historical data to infer potential solutions based upon the data distribution.

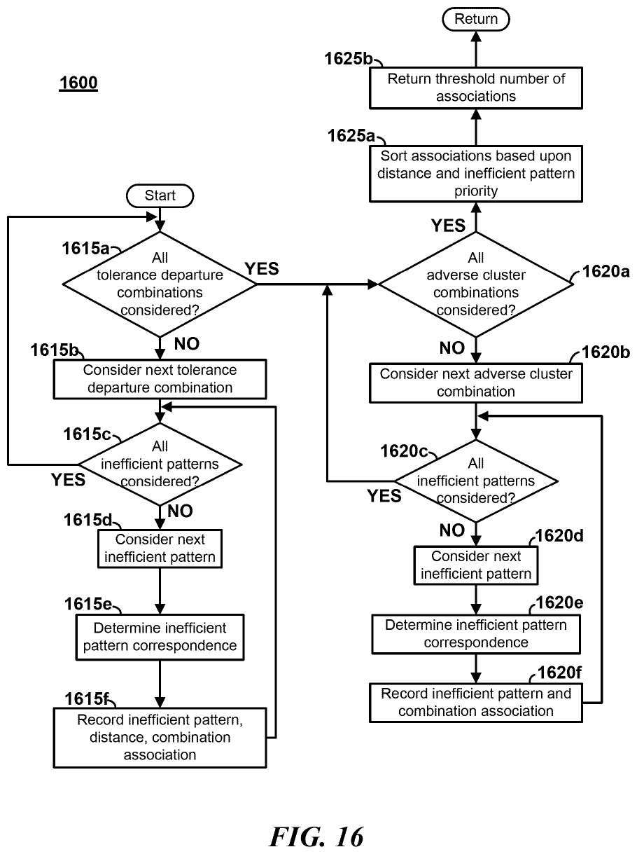

As another example, FIG. 16 is flow diagram illustrating various operations in an example process 1600 for providing coaching feedback based upon the determined metric values, as may be implemented in some embodiments. While focusing on relations between metric values and adverse/inefficient patterns in this example, one will appreciate variations that instead determine relations to desirable/efficient patterns (with corresponding remediations when the metrics depart too far from these preferred states). Similarly, in some embodiments, metrics and groups of metrics may be directly compared to known patterns without first identifying tolerance departures and cluster distances, as in the example process 1600.

Here, at blocks 1615a and 1615b, the system may iterate over all the previously identified tolerance departures (e.g., as determined at block 1510c) for the groupings of one or more metric results and consider whether they correspond with a known inefficient pattern at block 1615c (e.g., taking an inner product with the metric values with a known inefficient vector). For example, a protracted “case open to patient in” duration in combination with certain delay 810c and case volume 810a values, may, e.g., be indicative of a scheduling inefficiency where adjusting the scheduling regularly resolves the undesirable state. Note that the metric or metrics used for mapping to inefficient patterns for remediation may, or may not, be the same as the metric or metrics, which departed from the tolerance (e.g., at block 1615a) or approached the undesirable clustering (e.g., at block 1620a), e.g., the latter may instead indicate that the former may correspond to an inefficient pattern. For example, an outlier in one duration metric from FIG. 5A may imply an inefficient pattern derived from a combinations of metrics from FIG. 8.

Accordingly, the system may iterate through the possible inefficient patterns at blocks 1615c and 1615d to consider how the corresponding metric values resemble the inefficient pattern. For example, the Euclidean distance from the metrics to the pattern may be taken at block 1615e. At block 1615f, the system may record the similarity (e.g., the distance) between the inefficient pattern and the metrics group associated with the tolerance departure.

Similarly, following consideration of the tolerance departures, the system may consider metrics score combinations with clusters near adverse/inefficient events (e.g., as determined at block 1515a) at blocks 1620a and 1620b. As was done previously, the system may iterate over the possible known inefficient patterns at blocks 1620c and 1620d, again determining the inefficient pattern correspondence to the respective metric values (which may or may not be the same group of metric values identified in the cluster association of block 1620a) at block 1620e (again, e.g., the Euclidean or other appropriate similarity metric) and recording the degree of correspondence at block 1620f.

Based upon the distances and correspondences determined at blocks 1615e and 1620e, respectively, the system may determine a priority ordering for the detected inefficient patterns at block 1625a. At block 1625b, the system may return the most significant threshold number of inefficient pattern associations. For example, each inefficient pattern may be associated with a priority (e.g., high priority modes may be those with a potential for causing a downstream cascade of inefficiencies, patient harm, damage to equipment, etc., whereas lower priority modes may simply lead to temporal delays) and presented accordingly to reviewers. Consequently, each association may be scored as a weighted sum of a similarity between the score metric values and metric values associated with inefficient pattern and then weighted by the severity/priority of the inefficient pattern. In this manner, the most significant of the possible failures may be identified and returned first to the reviewer. The iterative nature of topology 450 may facilitate reconsideration and reweighting of the priorities for process 1600 as reviewers observe the impact of the proposed feedback over time. Similarly, the iterations may provide opportunities to identify additional remediation and inefficient pattern correspondences.

### Example GUI Nonoperative Metrics Analysis Feedback Elements

Presentation of the analysis results, e.g., at block 910l, may take a variety of forms in various embodiments. For example, FIG. 17 is a schematic representation of GUI elements in a quick review dashboard interface for nonoperative metrics review as may be implemented in some embodiments. In this example GUI 1705, selectors 1710a-d are provided for the user to select the temporal range of nonoperative period performance data that they wish to analyze. In this example, the user has selected to review the data captured during the past year. Following such a temporal selection, a “Nonoperative Metrics” region, a “Case Mix” region, and a “Metadata” region may be populated with values corresponding to the nonoperative periods for the selected range of data.

The “Case Mix” region may provide a general description of the data filtered from the temporal selection. Here, for example, there are 205 total cases (nonoperative periods) under consideration as indicated by label 1715a. A decomposition of those 205 cases is then provided by type of surgery via labels 1715b-d (specifically, that of the 205 nonoperative periods, 15 were associated with preparation for open surgeries, 180 with preparation for a robotic surgery, and 10 with preparation for a laparoscopic surgery). The nonoperative periods under consideration may be those occurring before and after the 205 surgeries, only those before, or only those after, etc., depending upon the user's selection.

The “Metadata” region may likewise be populated with various parameters describing the selected data, such as the number of ORs involved (8 per label 1720a), the number of specialties (4 per label 1720b), the number of procedure types (10 per label 1720c) and the number of different surgeons involved in the surgeries (27 per label 1720d).

Within the “Nonoperative Metrics” region, a holistic composite score, such as an ORA score, may be presented in region 1725a using the methods described herein (e.g., as described with respect to FIG. 8). Regions 1725b-f may show corresponding statistics for the intervals of FIG. 5A, here, values for various intervals of FIG. 5A.

Some embodiments may also present scoring metrics results comprehensively, e.g., to allow reviewers to quickly scan the feedback and to identify effective and ineffective aspects of the nonoperative theater performance. For example, FIG. 18A is a schematic representation of a GUI element 1805 as may be used for global quick review feedback in some embodiments. Specifically, individual metrics score values, composite metric scores, departures from tolerances, nearness to desirable or undesirable clustering, etc. may be indicated in a numerical region 1805d. The name of the metrics, etc., may be indicated in the name region 1805a and a desired feedback in region 1805b. A quick review icon 1805e may also be included to facilitate ready identification of the nature of the numerical feedback. A quality relation arrow region 1805c may be used to indicate whether the numerical value in region 1805d is above or below an operational point or tolerance, or trending upward or downward over time, and whether this is construed as indicative of improving or decreasing efficiency.

Specifically, FIG. 18B is a schematic representation of arrow elements as may be used in the quality relation arrow region 1805c of FIG. 18A in some embodiments. The arrow may be, e.g., color-coded to indicate whether the value is efficient (e.g., green) or inefficient (e.g., red). Thus, a rising arrow 1810a may indicate that the value in region 1805d is above a lower bound (e.g., when an idle time following successful completion of a task has increased above a historical average). Similarly, the falling arrow 1810b may indicate that the value in region 1805d is below an upper bound (e.g., when a preparation time has decreased below a historical average). Conversely, a falling arrow 1810c may indicate that the value in region 1805d is below a desired minimum value (e.g., when a number of personnel ready for a required step is below a historical average). Similarly, the rising arrow 1810d may indicate that the value in region 1805d is above a desired upper bound (e.g., when a preparation time has increased beyond a historical average).

By associating relational value both with the arrow direction and highlighting (such as by color, bolding, animation, etc.), reviewers may readily scan a large number of values and discern results indicating efficient or inefficient feedback. Highlighting may also take on a variety of degrees (e.g., alpha values, degree of bolding, frequency of an animation, etc.) to indicate a priority associated with an efficient or inefficient value. For example, FIG. 18C is a schematic representation of GUI elements in a quick review feedback interface 1820 as may be used in some embodiments. Here, the individual quick review feedbacks (instances of the element 1805) may be arranged in a grid and sized so that the reviewer may perceive multiple items at one time. Each element may be selectable, presenting details for the value determination, including, e.g., the corresponding historical data, theater-wide data, intermediate metrics calculation results, etc. One will appreciate that the figure is merely schematic and each “Action” or “Feedback” text may be replaced with one of the metrics described herein (e.g., a duration of intervals from FIGS. 5A-C) and remediations, respectively (though in some configurations the feedback may be omitted from all or some of the elements).

FIG. 19A is a plot of example analytic values as acquired in connection with a prototype implementation of an embodiment. Specifically, FIG. 19A shows results following processing for various of the intervals of FIG. 5A. Here, an outlier value 1905a clearly indicates a deviation in the “skin close to patient out” interval from the median duration of ˜10 minutes (taking instead approximately ˜260 minutes). FIG. 19B is similarly a plot of example operating room analytic values as acquired in connection with a prototype implementation of an embodiment. Here, standard deviation intervals may be shown to guide the reviewer in recognizing outlier values (e.g., whether they reflect a longer or shorter duration than the standard deviation interval).

### Computer System

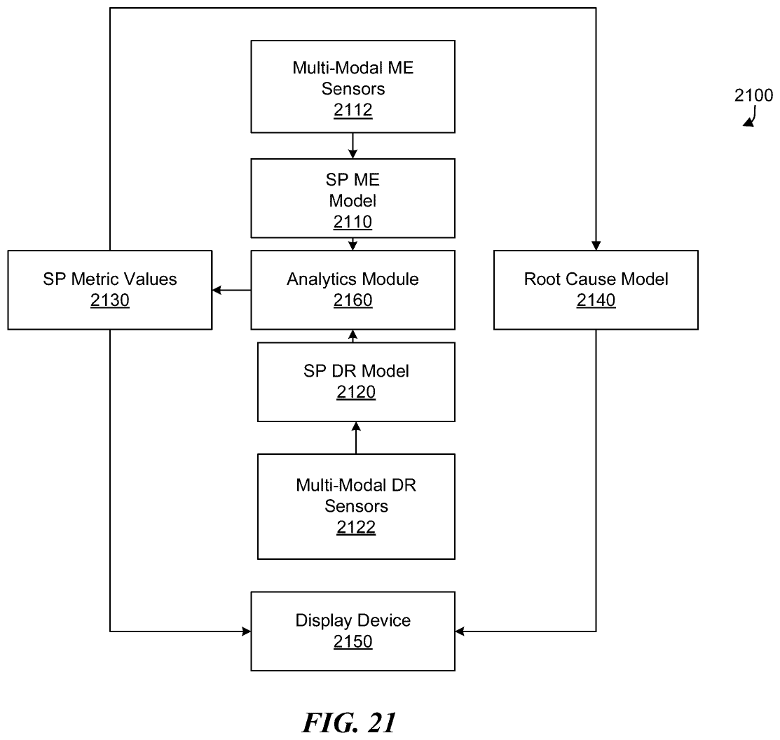

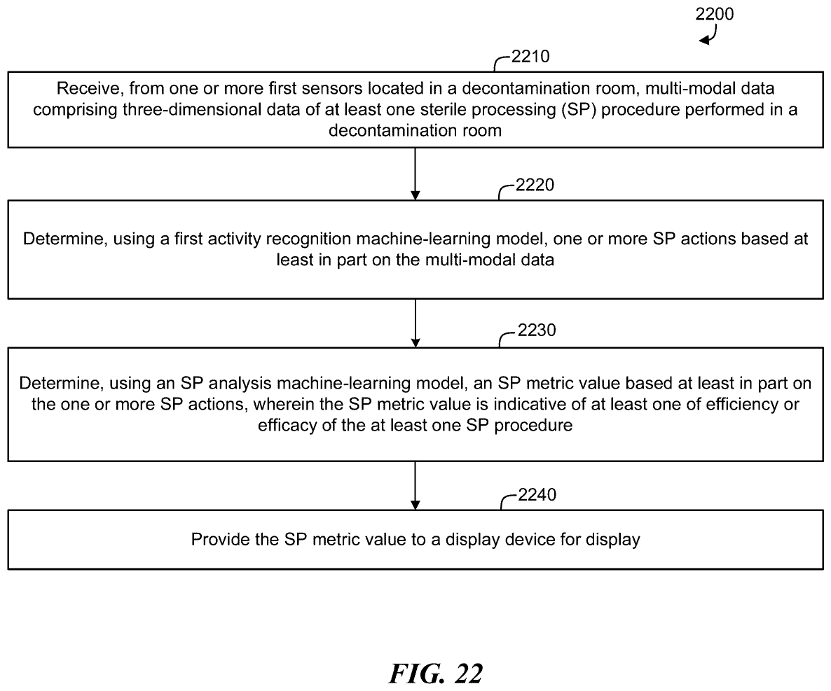

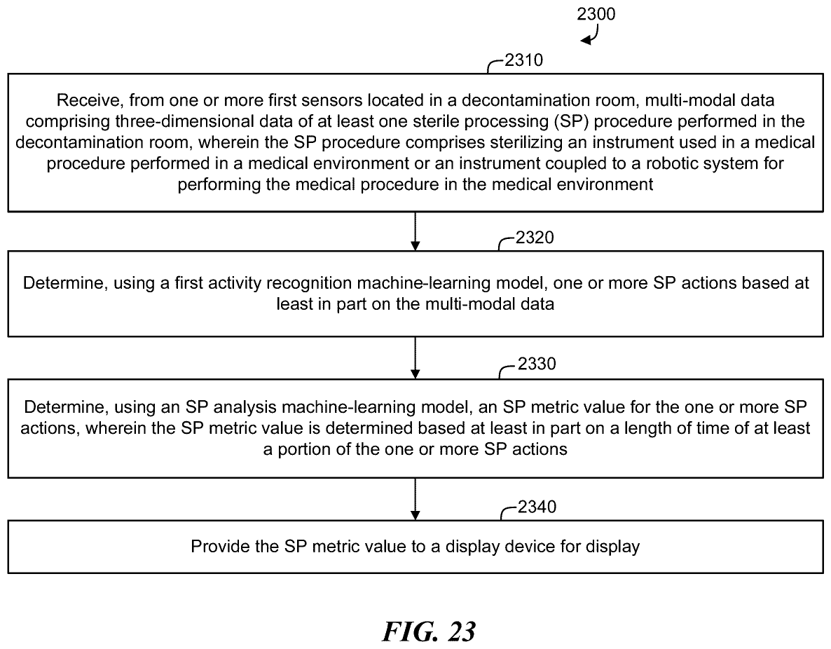

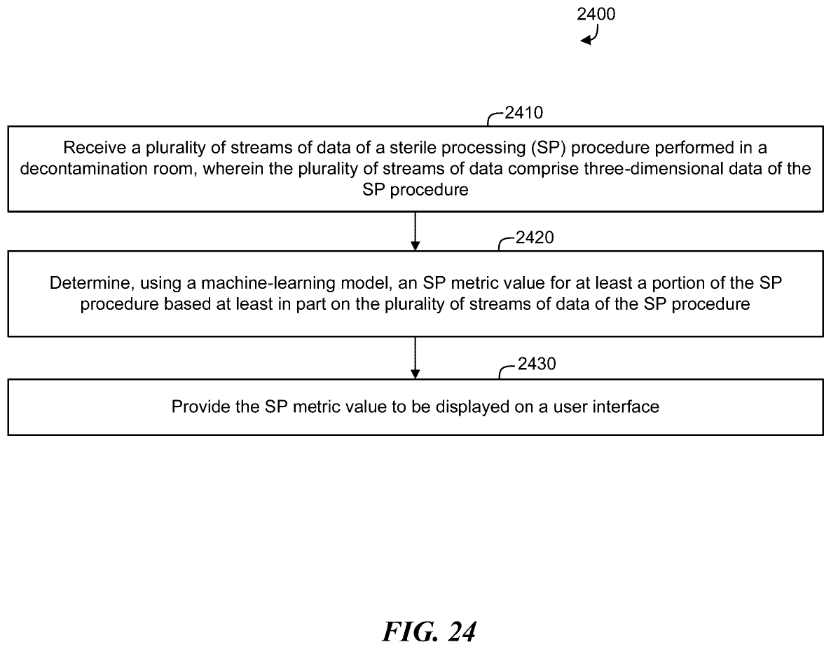

FIG. 20 is a block diagram of an example computer system 2000 as may be used in conjunction with some of the embodiments. In some examples, each of the processing systems 450b can be implemented using the computing system 2000. In some examples, the application 450f can be executed using the computing system 2000. In some examples, the system 2100 of FIG. 21 may be implemented at least in part using the computing system 2000. In some examples, the method 2200 of FIG. 22, the method 2300 of FIG. 23, and/or the method 2400 of FIG. 24 may be performed at least in part by the computing system 2000. The computing system 2000 may include an interconnect 2005, connecting several components, such as, e.g., one or more processors 2010, one or more memory components 2015, one or more input/output system(s) 2020, one or more storage systems 2025, one or more network adaptors 2020, etc. The interconnect 2005 may be, e.g., one or more bridges, traces, busses (e.g., an ISA, SCSI, PCI, I2C, Firewire bus, etc.), wires, adapters, or controllers.

The one or more processors 2010 may include, e.g., a general-purpose processor (e.g., x86 processor, RISC processor, etc.), a math coprocessor, a graphics processor, etc. The one or more memory components 2015 may include, e.g., a volatile memory (RAM, SRAM, DRAM, etc.), a non-volatile memory (EPROM, ROM, Flash memory, etc.), or similar devices. The one or more input/output device(s) 2020 may include, e.g., display devices, keyboards, pointing devices, touchscreen devices, etc. The one or more storage devices 2025 may include, e.g., cloud-based storages, removable Universal Serial Bus (USB) storage, disk drives, etc. In some systems memory components 2015 and storage devices 2025 may be the same components. Network adapters 2020 may include, e.g., wired network interfaces, wireless interfaces, Bluetooth™ adapters, line-of-sight interfaces, etc.

One will recognize that only some of the components, alternative components, or additional components than those depicted in FIG. 20 may be present in some embodiments. Similarly, the components may be combined or serve dual-purposes in some systems. The components may be implemented using special-purpose hardwired circuitry such as, for example, one or more ASICs, PLDs, FPGAs, etc. Thus, some embodiments may be implemented in, for example, programmable circuitry (e.g., one or more microprocessors) programmed with software and/or firmware, or entirely in special-purpose hardwired (non-programmable) circuitry, or in a combination of such forms.

In some embodiments, data structures and message structures may be stored or transmitted via a data transmission medium, e.g., a signal on a communications link, via the network adapters 2020. Transmission may occur across a variety of mediums, e.g., the Internet, a local area network, a wide area network, or a point-to-point dial-up connection, etc. Thus, “computer readable media” can include computer-readable storage media (e.g., “non-transitory” computer-readable media) and computer-readable transmission media.

The one or more memory components 2015 and one or more storage devices 2025 may be computer-readable storage media. In some embodiments, the one or more memory components 2015 or one or more storage devices 2025 may store instructions, which may perform or cause to be performed various of the operations discussed herein. In some embodiments, the instructions stored in memory 2015 can be implemented as software and/or firmware. These instructions may be used to perform operations on the one or more processors 2010 to carry out processes described herein. In some embodiments, such instructions may be provided to the one or more processors 2010 by downloading the instructions from another system, e.g., via network adapter 2020.

For clarity, one will appreciate that while a computer system may be a single machine, residing at a single location, having one or more of the components of FIG. 20, this need not be the case. For example, distributed network computer systems may include multiple individual processing workstations, each workstation having some, or all, of the components depicted in FIG. 20. Processing and various operations described herein may accordingly be spread across the one or more workstations of such a computer system. For example, one will appreciate that a process amenable to being run in a single thread upon a single workstation may instead be separated into an arbitrary number of sub-threads across one or more workstations, such sub-threads then run in serial or in parallel to achieve a same, or substantially similar, result as the process run within the single thread. Similarly, one will appreciate that while a non-transitory computer readable medium may stand alone (e.g., in a single USB storage device), or reside within a single workstation (e.g., in the workstation's random access memory or disk storage), such a medium need not reside at a single geographic location, but may include, e.g., multiple memory storage units residing across geographically separated workstations of a computer system in network communication with one another or across geographically separated storage devices.

### Generating Metric Values for Sterile Processing

FIG. 21 is a schematic block diagram illustrating an example system 2100 for generating SP metric values 2130 based at least in part on multi-modal data from a DR, according to various embodiments. The system 2100 includes an SP DR model 2120 that receives multi-modal data from multi-modal DR sensors 2122 (e.g., first sensors). The multi-modal DR sensors 2122 may capture three-dimensional data of SP procedures performed in the DR. In an example, the multi-modal DR sensors 2122 include a camera and a depth sensor in the DR to capture three-dimensional data of SP procedures performed in the DR. The DR sensors 2122 may be similar to the theater-wide sensors described herein, including the sensors 170a and 170c, the sensors capturing the theater-wide sensor perspective 205, the sensors 220a, 220b, 220c, 1335, 1365, and so on, except that the DR sensors 2122 capture the data within the DR rather than in the medical environment where the medical procedure is performed (e.g., OR).

In some examples, the DR sensors 2122 can include exocentric sensors (similar to the theater-wide sensor) and/or egocentric sensors. For example, one or more exocentric sensors (e.g., fixed on a wall or ceiling of the DR) can be configured to capture a “third person view” (TPV) of the DR, and one or more egocentric sensors can be configured to capture corresponding “first person views” (FPVs) with respect to SP personnel in the DR. An egocentric sensor can be a wearable sensor worn by or affixed to corresponding SP personnel in the DR. The exocentric sensors and the egocentric sensors, alone or in combination, can capture multi-modal data (e.g., depth data and image data) of the DR from their respective viewpoints. For example, an exocentric sensor can provide a TPV of the DR from a corner or wall of the DR, and a wearable sensor can provide an FPV of the DR from a head-worn sensor pose aligned with a field of view of the SP personnel. Thus, the exocentric sensors can each provide a distinct exocentric view independent of any individual in the DR, and the egocentric sensors can each provide a distinct egocentric view that corresponds to a viewpoint of a specific individual in the DR throughout an SP procedure performed in the DR. The inclusion of egocentric sensors allow capturing of a wholistic view of the DR and the SP procedure by capturing views that exocentric sensors are unable to capture due to occlusion.

The SP DR model 2120 (e.g., the first activity recognition algorithm or the first activity recognition machine-learning model) receives as input the multi-modal sensor data from the multi-modal DR sensors 2122 to identify activities performed in the DR. The SP DR model 2120 may identify the activities and when the activities are performed by adding timestamps and/or a label of the activity to the multi-modal sensor data or by recording timestamps corresponding to identified activities in the multi-modal data. The identified activities can correspond to parts, steps, and sub-steps of an SP procedure. For example, the identified activities may include one or more time stamps or labels for each of a part, step, or sub-step of an SP procedure. The parts of the SP procedures may include portions of the SP procedure such as receiving, washing, sanitizing, and packing. Each part of the SP procedures may include one or more steps, such as disassembly, rinsing, scrubbing, ultrasonic cleaning, and washer disinfection for a part such as washing. Each step may include one or more sub-steps such as mechanical cleaning of a portion of an instrument, flushing, lubricating, and washer loading for a step such as rinsing. The SP DR model 2120 may identify the activities for one or more of the parts, steps, and sub-steps of an SP procedure.

In some implementations, the SP DR model 2120 identifies the activities based at least in part on a location of the activities. In an example, the SP DR model 2120 may infer that actions performed in a vicinity of a washer correspond to activities of placing instruments in the washer to wash the instruments. In an example, the SP DR model 2120 may infer that actions performed in an intake area of the DR correspond to activities of initial rinses of instruments. In some implementations, the SP DR model 2120 (and/or the SP ME model 2110) includes a classification algorithm or model for identifying instruments (e.g., scalpel, endoscope), SP equipment (e.g., washer, autoclave), and/or personnel in order to identify the activities, instruments, and/or personnel performing the activities.

The SP DR model 2120 may be trained using a supervised training process, a self-supervised training process and/or a semi-supervised training process. In an example, the SP DR model 2120 may be trained by comparing timestamps and/or activity labels generated by the SP DR model 2120 to labeled timestamps and/or activity labels to decrease a distance between the generated timestamps and/or activity labels and the labeled timestamps and/or activity labels. In an example, the SP DR model 2120 may be trained by comparing historical calculations of SP procedure duration lengths for historical time periods (e.g., output of the SP DR model 2120) to ground truth including actual SP procedure duration lengths for the historical time periods. The actual SP procedure duration lengths, or the labeled SP procedure duration lengths (the ground truth) may be determined by a user labeling the SP procedure duration lengths. In some examples, the actual SP procedure duration lengths or the labeled SP procedure duration lengths has an SP metric value determined by a user and labeled with respect to a duration of an SP procedure or a date and time. A loss may be calculated based on a difference between the calculated SP procedure duration lengths and the actual SP procedure duration lengths. In an example, the loss is an MSE. The loss may be used to update one or more weights, parameters, and/or biases of the SP DR model 2120. Multiple iterations of generating the timestamps and/or activity labels, comparing the calculated timestamps and/or activity labels to labeled timestamps and/or activity labels, calculating a loss, and updating the SP DR model 2120 may be performed to improve the SP DR model 2120.

In some implementations, different aspects of the SP DR model 2120 can be trained using different training approaches. In an example, a first algorithm of the SP DR model 2120 may be trained using a supervised training process and a second algorithm of the SP DR model 2120 may be trained using a self-supervised training process.

An analytics module 2160 (e.g., an SP analysis machine-learning model, an SP analysis model, or a collection of statistical algorithms) may determine the SP metric values 2130 based on the identified activities identified by the SP DR model 2120. In some implementations, the analytics module 2160 determines the SP metric values 2130 based on the multi-modal data labeled with the time stamps and/or the activities. In some implementations, the analytics module 2160 determines the SP metric values 2130 based on the time stamps and/or activity identifiers.

In an example, the SP metric values 2130 may correspond to a length of a duration of an SP procedure, a length of a duration of a part of the SP procedure, a length of a duration of a step of the SP procedure, and a length of a duration of a sub-step of the SP procedure. That is, the SP metric values 2130 may include temporal metrics for the SP procedures. In some examples, an SP metric value 2130 includes or corresponds to a length of a duration of a part, step, or sub-step of the SP procedure. In some examples, an SP metric value 2130 includes or corresponds to an average or standard deviation of one or more lengths of the duration of the part, step, or sub-step performed in the same DR, in a hospital, in DRs in a region or a country, and so on. In some examples, an SP metric value 2130 includes or can be determined based on comparing of the actual length of a duration of a part, step, or sub-step with statistical or historical data such as 1) one or more target lengths of the duration of the part, step, or sub-step, or 2) historical averages or standard deviations of one or more actual lengths of the duration of the part, step, or sub-step performed previously in the same DR, in a hospital, in DRs in a region or a country, and so on. In some examples, an SP metric value 2130 includes or corresponds to lengths of durations of parts, steps, or sub-steps performed by different personnel. In some examples, an SP metric value 2130 includes or can be determined based on comparing a processing speed (e.g., efficiency) and efficacy for different SP personnel. In some examples, an SP metric value 2130 includes a temporal metric value for each of a plurality of instruments used in one or more SP procedures. In some examples, an SP metric value 2130 includes a turnaround time for each of a plurality of instruments used in one or more SP procedures. The turnaround time for an instrument may be defined as a time from when an instrument is delivered to the DR to a time when the instrument is ready (e.g., cleaned, sterilized, and packed) for use. In some examples, the SP metric values 2130 include average turnaround times for each type of instrument of the plurality of instruments used in one or more SP procedures. In this way, the SP metric values 2130 can indicate a length of time that an instrument is in SP (or a part, step, or sub-step thereof) and a length of time that an instrument can be expected to spend in SP between medical procedures.

The SP DR model 2120 may include a segmentation algorithm for identifying the parts, steps, and/or sub-steps of SP procedures, similar to the processing systems 450b to perform automated inference 450c of FIG. 4, or the process described in FIG. 10.

The SP metric values 2130 may indicate an adherence to instructions (e.g., IFUs) for performing an SP procedure. In an example, the SP metric values 2130 can indicate whether an individual part, step, or sub-step was performed, and/or whether the part, step, sub-step was performed correctly. In an example, the SP metric values 2130 include an indication of a percentage of an SP procedure that was performed correctly (e.g., a percentage of the parts, steps, sub-steps that were performed correctly). In some implementations, the instructions for performing the SP procedure include amounts of time for each part, step, and/or sub-part. That is, a length of a duration of a part, step, or sub-step that is consistent with the instruction can receive an SP metric values 2130 indicating satisfactory score, and a length of a duration of a part, step, or sub-step that is inconsistent with the instruction can receive an SP metric values 2130 indicating an unsatisfactory score. In an example, an instrument may need to be soaked in a solution for at least twenty minutes, but no more than thirty minutes. In this example, the SP metric values 2130 may indicate whether the instrument was soaked according to the instructions and/or an amount of time the instrument was soaked. In this example, the SP metric values 2130 may indicate a satisfactory score, or correct completion of the soaking if the instrument was soaked between twenty to thirty minutes and an unsatisfactory score, or incorrect completion of the soaking if the instrument was soaked less than twenty minutes or more than thirty minutes. In this example, the unsatisfactory score may be more unsatisfactory if the instrument was soaked less than twenty minutes than if the instrument was soaked more than thirty minutes. In this example, the unsatisfactory score may be more unsatisfactory the farther from the acceptable range the time of soaking is.

The SP metric values 2130 may include or indicate a throughput for the DR. The throughput may include a number of instruments processed in the DR in a period of time, or a number of instruments that pass through the DR in the period of time. In an example, the SP metric values 2130 include a daily processing throughput for the DR such as an average number of instruments processed daily. In an example, the SP metric values 2130 include a throughput for each instrument or type of instrument of a plurality of instruments. In an example, the SP metric values 2130 include a throughput for one or more of a shift, team, SP personnel, DR, hospital, region, country, and so on. The SP metric values 2130 may include benchmarks for the throughput for the DR.

In some implementations, the SP metric values 2130 include ergonomic statistics for SP personnel. In an example, the SP metric values 2130 include a shift duration for the SP personnel and a rest/activity ration for the SP personnel. In an example, the SP metric values 2130 include a total time standing for the SP personnel. In an example, the SP metric values include a total time spent scrubbing, washing, rinsing, and/or packing for the SP personnel. In this way, repetitive tasks or physically demanding tasks performed by the SP personnel can be tracked in order to reduce injury.

The SP metric values 2130 may include utilization statistics for SP equipment. The SP equipment may include, but is not limited to, an ultrasonic cleaner, a washer disinfector, and an autoclave. In an example, the SP metric values 2130 include utilization time and idle time for each piece of SP equipment. In an example, the SP metric values 2130 indicate a percentage of time utilized for each piece of SP equipment. The SP metric values 2130 may include amounts of time SP personnel interact with the SP equipment and amounts of time the equipment operates without SP personnel interaction. In an example, the SP metric values 2130 indicate that an autoclave is loaded in three minutes and then operates independently (without additional interaction) for thirty minutes.

The SP metric values 2130 may include adverse events. The adverse events may include mishandled I&A or contamination of I&A. The adverse events may include equipment malfunction and/or human error. The SP metric values 2130 may include historical adverse event statistics. In this way, the SP metric values 2130 may indicate recurring or persistent issues with equipment or personnel. In an example, the SP metric values 2130 indicate that an SP technician causes contamination of medical instruments during a specific SP procedure, allowing for intervention, such as additional training.

The SP metric values 2130 may include and/or may be used to determine an efficiency or an efficacy of the SP procedures. The efficiency of the SP procedures may be based on how many personnel performed the SP procedures, how many hours the SP procedures required, how many hours worked the SP procedures required, and/or the daily throughput of the DR. The efficacy of the SP procedures my include statistics of adherence to the predefined instructions for the SP procedures and/or statistics of instrument status after the SP procedures.

The SP DR model 2120 may determine one or more actions and/or objects from the multi-modal data from which the analytics module 2160 generates the SP metric values 2130. The SP DR model 2120 may identify an instrument, a type of instrument, a piece of SP equipment, and/or SP personnel. The SP DR model 2120 may identify actions such as movement of instruments and/or the parts, steps, and sub-steps of the SP procedures. In an example, the SP DR model 2120 identifies an instrument delivered to the DR and tracks the instrument through the SP procedures. In some implementations, the SP DR model 2120 (and/or the SP ME model 2110) includes a classification algorithm or model for classifying the instruments. In some implementations, the SP DR model 2120 correlates other tracking data (e.g., barcode scans, RFID tag scans, manual tracking of instruments) with the identification of the instrument to track the instrument through the SP procedures. In this way, the analytics module 2160 can generate the SP metric values 2130 according to the SP of each instrument.

The SP DR model 2120 may include an activity recognition model (e.g., a first activity recognition algorithm) trained using data from the DR or another DR. The SP DR model 2120 may generate additional data based on the multi-modal data from which the analytics module 2160 generates the SP metric values 2130. In an example, the SP DR model 2120 adds timestamps to the multi-modal data corresponding to the start and end of different actions or the parts, steps, and sub-steps of the SP procedures. In an example, the SP DR model 2120 generates a data record based on time stamps in the multi-modal data corresponding to the start and end of different actions or the parts, steps, and sub-steps of the SP procedures. In an example, the SP DR model 2120 generates bounding boxes corresponding to instruments, objects, and/or personnel in the multi-modal data.

The analytics module 2160 may be trained using a supervised training process, a self-supervised training process and/or a semi-supervised training process. In an example, the analytics module 2160 may be trained by comparing the SP metric values 2130 generated by the analytics module 2160 to labeled metrics to decrease a distance between the SP metric values 2130 and the labeled metrics. In an example, the analytics module 2160 may be trained by comparing historical calculations of the SP turnaround times for historical time periods to actual SP turnaround times for the historical time periods. A loss may be calculated based on a difference between the calculated SP turnaround times and the actual SP turnaround times. In an example, the loss is an MSE. The loss may be used to update one or more weights, parameters, and/or biases of the analytics module 2160. Multiple iterations of generating the SP metric values 2130, comparing the calculated SP turnaround times to labeled SP metrics, calculating a loss, and updating the SP DR model 2120 may be performed to improve the analytics module 2160.

In some implementations, different aspects of the analytics module 2160 can be trained using different training approaches. In an example, a first algorithm of the analytics module 2160 may be trained using a supervised training process and a second algorithm of the analytics module 2160 may be trained using a self-supervised training process.

In some implementations, the analytics module 2160 includes a collection of statistical algorithms that compute relevant statistics of the SP metric values 2130. In this way, the analytics module 2160 can calculate the SP metric values 2130 without including a machine-learning model or can compute SP metric values in addition to those generated by using machine-learning models. As discussed herein, other embodiments of the analytics module 2160 include machine-learning models. In an example, the analytics module 2160 includes an algorithm that calculates a duration of an SP procedure based on time stamps and labels for the SP procedure determined by the SP DR model 2120. The analytics module 2160 may select an algorithm from the collection of statistical algorithms for computing a statistic (i.e., metric) of the SP metric values 2130. The analytics module 2160 may select the algorithm based on activity identifiers determined by the SP DR model 2120. In an example, the analytics module 2160 may select an autoclave idle time algorithm based on activity identifiers associated with an autoclave or with an SP procedure utilizing the autoclave. The analytics module 2160 may identify inputs to the collection of statistical algorithms based on the activity identifiers and/or the time stamps determined by the SP DR model 2120. In an example, the analytics module 2160 determines that time stamps associated with an activity identifier for scrubbing an endoscope should be used as input for an algorithm that calculates total time for SP procedures and/or for an algorithm that calculates total scrubbing time for different SP personnel. In an example, the analytics module 2160 determines that an activity is input for an algorithm that calculates total packing time (after washing, cleaning, and sterilizing), based on time stamps for the activity occurring after an autoclave completion time.

In some implementations, the system 2100 includes an SP ME model 2110 (e.g., the second activity recognition algorithm or the second activity recognition machine-learning model) that receives multi-modal data from multi-modal ME sensors 2112 (e.g., the second sensors). The multi-modal ME sensors 2112 may capture three-dimensional data of SP procedures, parts, steps, and/or sub-steps performed in the ME. In an example, an initial step of an SP procedure may be wiping or rinsing an instrument, which is performed in the ME. Any part, step, or sub-step of the SP procedure performed in the ME is referred to as an medical environment action of the SP. In an example, an initial step of an SP procedure may be placing an instrument in containers in the ME to be sent to the DR. In an example, the multi-modal ME sensors 2112 include a camera and a depth sensor in the DR to capture three-dimensional data of SP procedures parts, steps, and/or sub-steps performed in the DR. Examples of the multi-modal ME sensors 2112 include the theater-wide sensors described herein, including the sensors 170a and 170c, the sensors capturing the theater-wide sensor perspective 205, the sensors 220a, 220b, 220c, 1335, 1365, and so on.

The multi-modal ME sensors 2112 may include exocentric sensors and/or egocentric sensors. For example, one or more exocentric sensors (e.g., fixed on a wall or ceiling of the ME) can be configured to capture a TPV of the ME, and one or more egocentric sensors can be configured to capture corresponding FPVs of the ME. An egocentric sensor can be a wearable sensor worn by or affixed to corresponding ME personnel in the ME. the exocentric sensors and the egocentric sensors, alone or in combination, can capture multi-modal data (e.g., depth data and image data) of the ME from their respective viewpoints. For example, an exocentric sensor can provide a TPV of the ME from a corner or wall of the ME, and the wearable sensors can provide an FPV from respective head-worn sensor pose aligned with a field of view of the ME personnel. Thus, the exocentric sensors can each provide a distinct exocentric view independent of any individual in the ME, and the egocentric sensors can each provide a distinct egocentric view that corresponds to a viewpoint of a specific individual in the ME throughout an SP procedure performed in the ME. Similar to the DR, the inclusion of egocentric sensors allows capturing of a wholistic view of the ME and the medical procedure by capturing views that exocentric sensors are unable to capture due to occlusion.

The SP ME model 2110 may be trained using a supervised training process, a self-supervised training process and/or a semi-supervised training process. In an example, the SP ME model 2110 may be trained by comparing the SP metric values 2130 generated by the SP ME model 2110 to labeled metrics to decrease a distance between the SP metric values 2130 and the labeled metrics. In an example, the SP ME model 2110 may be trained by comparing historical determinations of SP actions in the ME (e.g., output of the SP ME model 2110) to ground truth including actual SP actions in the ME, or labeled SP actions in the ME for the historical time periods. The actual SP actions in the ME, or the labeled SP actions in the ME (the ground truth) may be determined by a user labeling the actions. In some examples, the actual SP actions in the ME or the labeled SP actions in the ME has an SP metric value determined by a user and labeled with respect to a duration of a medical procedure or a date and time. A loss may be calculated based on a difference between the determined SP actions and the actual SP actions. In an example, the loss is an MSE. The loss may be used to update one or more weights, parameters, and/or biases of the SP ME model 2110. Multiple iterations of determining SP ME actions, comparing the determined SP ME actions to labeled SP ME actions, calculating a loss, and updating the SP ME model 2110 may be performed to improve the SP ME model 2110.

In some implementations, different aspects of the SP ME model 2110 can be trained using different training approaches. In an example, a first algorithm of the SP ME model 2110 may be trained using a supervised training process and a second algorithm of the SP ME model 2110 may be trained using a self-supervised training process.

In some implementations, the SP ME model 2110 and the SP DR model 2120 can include similar algorithms or models to each other (e.g., activity recognition, segmenting, classification), and/or similar algorithms or models to the processing systems 450b to perform automated inference 450c, as described in FIG. 4, but tailored for their respective use cases. In an example, the SP ME model 2110 and the SP DR model 2120 are based on a common model, but are trained using ME data and DR data, respectively.

The analytics module 2160 may receive as input the output of the SP ME model 2110 and the SP DR model 2120. The analytics module 2160 may combine or correlate the output of the SP ME model 2110 with the output of the SP DR model 2120 to generate the SP metric values 2130. In an example, an action detected by the SP ME model 2110 in the ME of placement of an instrument in a container to be sent to the DR can be combined with an action detected by the SP DR model 2120 in the DR of washing of the instrument to track the instrument from the ME through the SP procedures, and/or to determine a time between use of the instrument in the ME to completion of the SP procedures for the instrument in the DR.

In some implementations, the analytics module 2160 may receive as input robotic system data and/or data derived from the robotic system data from a robotic system (e.g., the patient side cart 130). The robotic system data (or system data) includes kinematics data of a robotic system, system events data of the robotic system, input received by the console of the robotic system from a user, and timestamps associated therewith. The robotic system data of a robotic system can be generated by the robotic system (e.g., in the form of a robotic system log) in its normal course of operations. For example, the kinematics data can indicate configuration(s) of one or more manipulators or manipulator assemblies of the robotic system 130 as well as the instruments attached or operatively coupled to the manipulators or manipulator assemblies over time throughout the medical procedure.

Furthermore, the system events data can be generated by the robotic system and can indicate system events of the robotic system. Examples of system events can include, for example, a docking event (e.g., in which manipulator arms are docked to cannulas inserted into a patient anatomy), operator (e.g., surgeon) head-in or head-out event (e.g., indicating a surgeon's head being present or absent at a viewer on a input or control console of the robotic system), an instrument attachment or removal event (e.g., indicating attachment or removal of an instrument, such as a medical instrument or an imaging instrument, on a manipulator of the robotic system, a tool exchange event), an instrument change event (e.g., indicating performance of an exchange of one instrument for another instrument for attachment on a manipulator on the robotic system), a draping-start event or a sterile adapter attachment event (e.g., which may indicate beginning of a sterile draping process), and the like. The robotic system can include one or more sensors (e.g., camera, infrared sensor, ultrasonic sensors, etc.), actuators, interfaces, consoles, that can output information used to detect such a system event. Although system events such as instrument attachment or removal event and instrument change event can explicitly inform the installation or removal of an instrument, which may be subjected to SP, other system events can also provide implicit indication of the installation, use, removal of an instrument. Such robotic system data can be in natural language (e.g., a string of characters).

In other words, the robotic system data can be indicative of one or more states of one or more components of the robotic system. Components of the robotic system 130 can include, but are not limited to, actuators on the robotic system and instruments coupled or attached to the actuators of the robotic system. For example, the robotic system data can include one or more data points indicative of one or more of an activation state (e.g., activated or deactivated), a position, or orientation of a component of the robotic system 130. For example, the robotic system data can be linked with or correlated with one or more medical procedures, one or more phases of a given medical procedure, one or more tasks of a given phase of a given medical procedure, and steps of the SP. For example, a type of robotic system data can be indicative of a position of one or more actuators of a given arm (or an instrument attached thereto) of the robotic system 130 at a given time or over a given time interval. Such time interval can be associated with a given part, step, or sub-step of an SP procedure. In some examples, the robotic system data can include a timeline based on timestamps of system events that are time-aligned.

The robotic system data can indicate which I&A were used in a medical procedure. In some implementations, the robotic system data may be correlated with the multi-modal data from the multi-modal ME sensors 2112 and/or the multi-modal DR sensors 2122. In this way, the analytics module 2160 can track I&A from use in the ME, through the SP procedures in the DR, to the completion of the SP procedures, and/or to subsequent use in the ME.

The system may include a display device 2150. The display device 2150 may display the SP metric values 2130. The display device 2150 may include multiple display devices for different people or roles. In an example, the display device 2150 includes a first display device for a hospital administrator on which is displayed a first subset of the SP metric values and a second display device for SP personnel on which is displayed a second subset of the SP metric values. The display device 2150 may include indications of a status of instruments being processed in the DR. In an example, the display device 2150 indicates a progress of the instruments through the SP procedures, a part, step, and/or sub-step of each instrument in the SP procedures, and/or an estimated time of availability of the instruments.

The display device 2150 may display information, videos, images, or guides on a correct method for performing an identified part, step, or sub-part. In an example, the display device 2150 indicates a current sub-step and displays a video demonstrating correct performance of the current sub-step. In an example, the display device 2150 indicates a current sub-step and displays a diagram of correct performance of the current sub-step. The display device 2150 may display a metric values of the SP metric values 2130 corresponding to a current part, step, and/or sub-step. In an example, the display device 2150 displays an average duration for a current part and step, a target duration for the current part, and a target duration for the current step.

The display device 2150 may include an interactive display, such as an interactive wall chart. Conventional DRs include static wall charts which include instructions for performing SP procedures. The display device 2150 may display an interactive wall chart that displays the instructions for performing the SP procedures, a progress of the SP procedures, a next step of the SP procedures, and/or specific instructions for a current part, step, and/or sub-step of the SP procedures. The display device 2150 may include various indicators for the equipment in the DR. In an example, the display device 2150 includes an interactive wall chart instructing SP personnel to use an autoclave in a next step, and a light adjacent the autoclave to guide the SP personnel to the autoclave. In an example, the display device 2150 includes an interactive wall chart instructing SP personnel to remove an instrument from an autoclave after twenty minutes, and a light adjacent the autoclave to indicate that the autoclave is sanitizing the instrument.

In some implementations, the display device 2150 may display a notification to indicate to SP personnel of completion of an SP procedure, adherence to IFUs, and/or a score for performance of the SP procedure. In an example, the notification indicates a percentage complete of the SP procedure and an indication of a next step. The display device 2150 may indicate that one or more steps need to be repeated to fully decontaminate medical instruments. The display device 2150 may indicate that one or more instruments have been recontaminated and need to be reprocessed. In some implementations, the display device 2150 displays a contamination level of one or more instruments. In an example, the display device 2150 displays a heat map of the DR showing generally where contaminated instruments are located.

The display device 2150 may include an augmented reality (AR) headset. The AR headset may provide an AR overlay for the SP personnel. In an example, the AR overlay indicates an instrument to be processed, equipment to process the instrument, and/or a part, step, or sub-step of the SP procedure. In an example, the AR overlay includes an indication of equipment availability. In an example, the AR overlay includes identifiers of I&A. In an example, the AR overlay includes a timer indicating how long a part, step, or sub-step should take and/or a remaining amount of time for the part, step, or sub-step. In an example, the AR overlay includes identifiers of equipment and use status of the equipment.

The system may include a root cause model 2140 (e.g., a root cause system). The root cause model 2140 may receive as input the SP metric values 2130, the output of the SP DR model 2120 (e.g., identified actions and/or objects), and/or the output of the SP ME model 2110 to generate a root cause analysis. The root cause analysis may include explanations of the SP metric values 2130 and/or recommendations to improve the SP metric values 2130. The root cause analysis may include a potential cause, support analytics, a recommendation, and a score for ranking one or more of the potential cause, support analytics, and recommendation. In an example, the root cause analysis includes a metric value of the SP metric values 2130, an explanation of the metric value, support analytics supporting the explanation, a recommendation, and a score indicating a confidence of the recommendation. The recommendations output by the root cause model 2140 may include one or more of training for SP staff, DR layout optimization, resource allocation to minimize equipment idle time, and optimized levels of staff and equipment. In an example, the root cause model 2140 may recommend providing additional training for SP staff for processing a specific instrument type. In an example, the root cause model 2140 may recommend improving the DR layout by moving a piece of equipment nearer another piece of equipment used in the same SP procedure. In an example, the root cause model 2140 may recommend increasing a number of staff to increase the throughput of the DR. In an example, the root cause model 2140 may recommend decreasing equipment count to improve the layout of the DR and/or to reduce equipment idle time. In an example, the root cause model 2140 may recommend adjusting a level of inventory to increase an efficiency of inventory use. In an example, the root cause model 2140 may recommend adjusting an assignment of SP procedure actions to SP personnel. In this example, the root cause model 2140 may recommend combining one or more actions to be performed by a single person or separating actions to be performed by different people.

The root cause model 2140 may provide the potential cause, support analytics, recommendation, and/or any scores to the display device 2150 for display. The root cause model 2140 may provide additional data and insight for the SP metric values 2130 displayed on the display device. In an example, the root cause model 2140 causes the displayed SP metric values 2130 to include the potential cause and recommendation. In an example, the root cause model 2140 causes the SP metric values 2130 to be displayed on the display device 2150 to an administrator with a recommendation of optimized personnel and equipment levels for the DR. In an example, the 2140 causes the SP metric values 2130 to be displayed on the display device 2150 in the DR to recommend to SP personnel to pay close attention to a particular sub-step of an SP procedure or to change an order of SP procedures, parts, steps, or sub-steps. In this way, the recommendations from the root cause model 2140 can be used to change various parameters of the DR, the SP procedures, and/or the behavior of the SP personnel.

In some implementations, the root cause model 2140 can store a mapping between a plurality of potential causes and a plurality of indications. In some examples, one indication can be mapped to multiple potential causes. In some examples, multiple indication can be mapped to one potential cause. In some examples, one indication can be mapped to one potential cause. The mapping can be predetermined offline based on historical benchmarks, expert opinions, and so on. The mapping may be updated based on updated data, metrics, and previously-implemented recommendations.

Accordingly, the root cause model 2140 can automate the discovery of workflow analysis and enables end-to-end root cause analysis. The root cause model 2140 removes the subjectivity and bias from consultation. Expert opinion can captured in design of the root cause model 2140 in the forms of mappings described herein. The root cause model 2140 can consider all potential indications and can provide a comprehensive list of potential inefficiencies and efficiencies, as opposed to an individual who can consider only a subset of such indications. The root cause model 2140 is customizable for individual users based on their roles and groups.

FIG. 22 is a flowchart diagram illustrating an example method 2200 for generating SP metric values including efficiency or efficacy of an SP procedure based on multi-modal data from a DR, according to various embodiments. The method 2200 may include more, fewer, or different operations than shown. The operations may be performed in the order shown, in a different order, or concurrently. The method 2200 may be performed by the system 2100 of FIG. 21.

At 2200, one or more processors receive, from one or more first sensors located in a DR, multi-modal data comprising three-dimensional data of at least one sterile processing (SP) procedure performed in a DR. An example of the first sensors are the multi-modal SP DR sensors 2122 of FIG. 21. In some implementations, the one or more first sensors include egocentric and exocentric sensors.

At 2220, the one or more processors determine, using a first activity recognition machine-learning model, one or more SP actions based at least in part on the multi-modal data. An example of the first activity recognition machine-learning model is the SP DR model 2120 of FIG. 21. Determining the one or more SP actions may include adding timestamps to the three-dimensional data associated with the one or more SP actions.

At 2230, the one or more processors determine, using an SP analysis machine-learning model, an SP metric value based at least in part on the one or more SP actions, wherein the SP metric value is indicative of at least one of efficiency or efficacy of the at least one SP procedure. An example of the SP analysis machine-learning model is the analytics module 2160 of FIG. 21. In some implementations, the SP analysis machine-learning model uses as input robotic system data corresponding to medical procedures using instruments for which SP is performed. In some implementations, the robotic system data indicates usage of the instruments. In some implementations, the SP analysis machine-learning model receives the robotic system data from a robotic system in an ME. In some implementations, the SP analysis machine-learning model tracks the instruments from usage in the ME to completion of the at least one SP procedure in the DR.

In some implementations, the SP metric value includes or is determined based at least in part on an SP turnaround time. In some implementations, the SP metric value includes or is determined based at least in part on SP turnaround times for each medical procedure of a plurality of medical procedures. In some implementations, the SP metric value includes or is determined based at least in part on SP turnaround times for each instrument type of a plurality of instrument types. In some implementations, the SP metric value includes one or more of SP equipment utilization, completion of steps of the at least one SP procedure, temporal metrics for the steps of the at least one SP procedure, and SP throughput.

In some implementations, the one or more processors generate, using the SP analysis machine-learning model, alerts based on detecting one or more of detecting mishandled instruments, contamination of instruments, and infection risk for SP staff.

In some implementations, the one or more processors generate one or more recommendations based on one or more of the SP metric value and the three-dimensional data of the at least one SP procedure. In some implementations, the one or more recommendations include one or more of training for SP staff, DR layout optimization, better resource allocation to minimize equipment idle time, and optimized staff and equipment levels.

At 2240, the one or more processors provide the SP metric to a display device for display. An example of the display device is the display device 2150 of FIG. 21. In some implementations, the display device is an interactive wall chart. In some implementations, the display device indicates a status of instruments being processed. In some implementations, the display device includes an AR headset. In some implementations, the display device includes information, videos, images, or guides on the correct method for performing an identified part, step, or sub-step along with an SP metric value for that part, step, or sub-part.

The method 2200 may include determining, by the one or more processors, using a second activity recognition machine-learning model, one or more medical environment (ME) actions based on second multi-modal data including second three-dimensional data of an ME. An example, of the second activity recognition machine-learning model is an activity recognition model included in the SP ME model 2110 of FIG. 21. The first activity recognition machine-learning model may be trained using DR data, and the second activity recognition machine-learning model may be trained using ME data

FIG. 23 is a flowchart diagram illustrating an example method 2300 for generating SP metric values based on multi-modal data from a DR, according to various embodiments. The method 2300 may include more, fewer, or different operations than shown. The operations may be performed in the order shown, in a different order, or concurrently. The method 2300 may be performed by the system 2100 of FIG. 21.

At 2310, one or more processors receive, from one or more first sensors located in a DR, multi-modal data comprising three-dimensional data of at least one sterile processing (SP) procedure performed in the DR, wherein the SP procedure comprises sterilizing an instrument used in a medical procedure performed in a medical environment or an instrument coupled to a robotic system for performing the medical procedure in the medical environment. An example of the one or more first sensors are the multi-modal SP DR sensors 2122 of FIG. 21.

At 2320, the one or more processors determine, using a first activity recognition machine-learning model, one or more SP actions based at least in part on the multi-modal data. An example of the first activity recognition machine-learning model is the SP DR model 2120 of FIG. 21.

At 2330, the one or more processors determine, using an SP analysis machine-learning model, an SP metric value for the one or more SP actions, wherein the SP metric value is determined based at least in part on a length of time of at least a portion of the one or more SP actions. An example of the SP analysis machine-learning model is the analytics module 2160 of FIG. 21.

At 2340, the one or more processors provide the SP metric value to a display device for display. An example of the display device is the display device 2150 of FIG. 21.

FIG. 24 is a flowchart diagram illustrating an example method 2400 for generating SP metric values based on multi-modal data from a DR, according to various embodiments. The method 2400 may include more, fewer, or different operations than shown. The operations may be performed in the order shown, in a different order, or concurrently. The method 2400 may be performed by the system 2100 of FIG. 21.

At 2410, one or more processors receive a plurality of streams of data of a sterile processing (SP) procedure performed in a DR, wherein the plurality of streams of data comprise three-dimensional data of the SP procedure.

At 2420, the one or more processors determine, using a machine-learning model, an SP metric value for at least a portion of the SP procedure based at least in part on the plurality of streams of data of the SP procedure. An example of the machine-learning model is the analytics module 2160 of FIG. 21.

At 2430, the one or more processors provide the SP metric value to be displayed on a user interface. An example of the display device is the display device 2150 of FIG. 21.

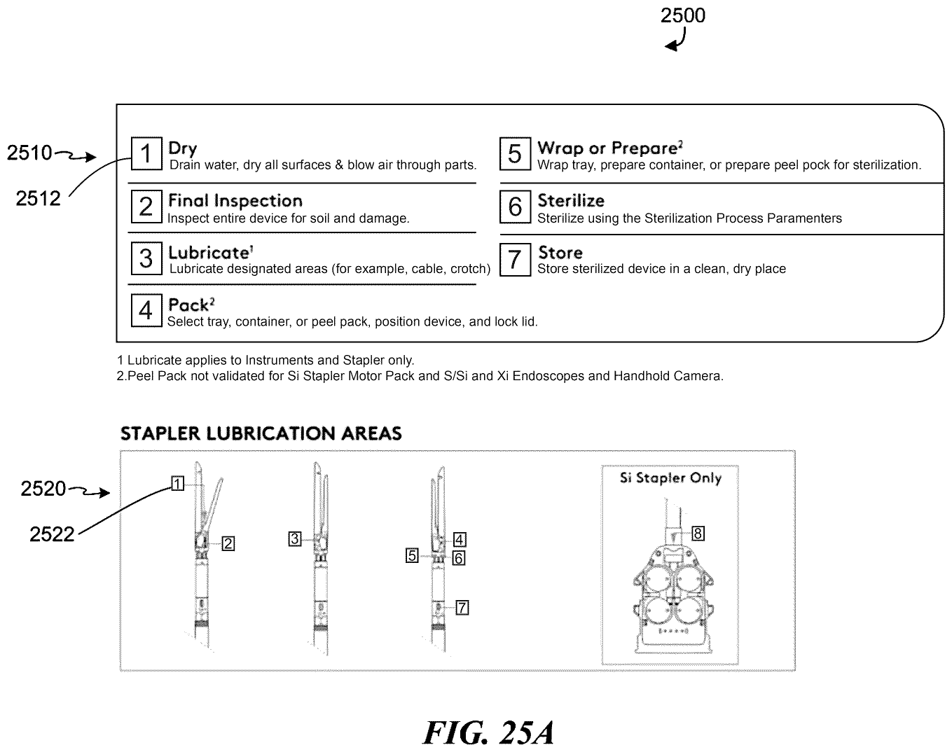

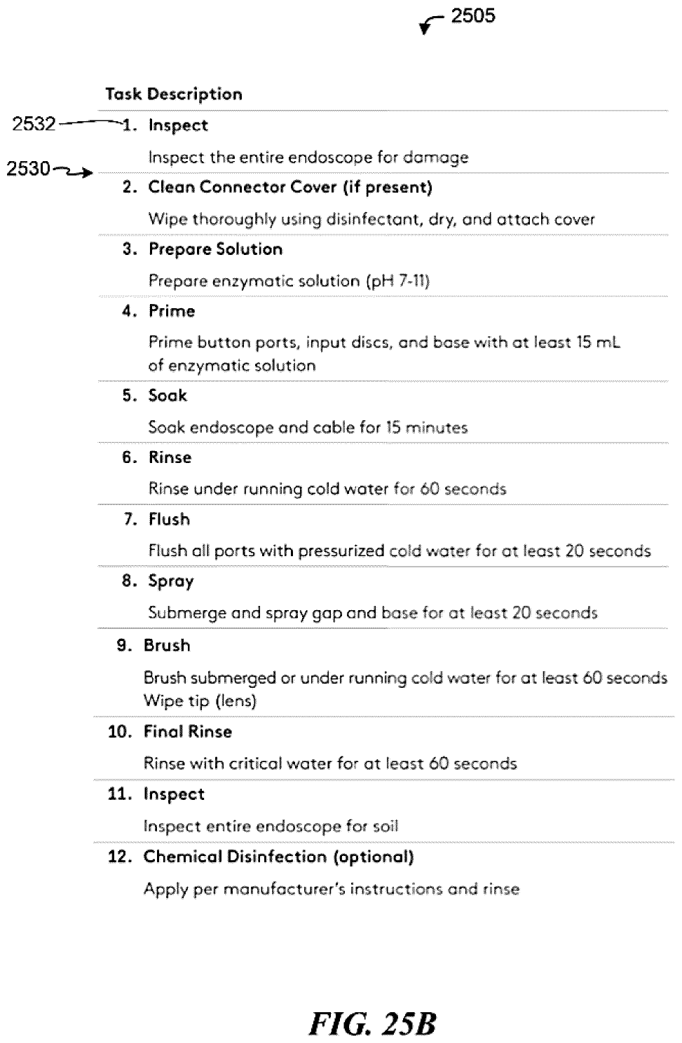

FIG. 25A is an example user interface 2500 showing instructions for an SP procedure, according to various embodiments. The instructions may include a description 2510 of one or more actions to be performed, or of parts, steps, or sub-steps of the SP procedure. The instructions may include one or more indicators 2512 associated with the actions to be performed or parts, steps, or sub-steps of the SP procedure. The one or more indicators 2512 may indicate a progress of the SP procedure and/or a completion of the actions to be performed or parts, steps, or sub-steps of the SP procedure. The instructions may include one or more diagrams 2520. The one or more diagrams 2520 may depict instruments to be processed. The one or more diagrams 2520 may include one or more indicators 2522 indicating parts or locations of the instruments to be processed.

The user interface 2500 may be interactive, such that selecting the one or more indicators 2512 causes the user interface 2500 to show more detailed instructions for the corresponding action to be performed, or part, step, or sub-step of the SP procedure. Selecting the one or more indicators 2522 may cause the user interface 2500 to provide more detailed instructions or explanations of the instruments, and/or the parts or locations of the instruments.

The user interface 2500 may be displayed on a display device such as an interactive wall chart, a computer monitor, and/or an AR headset. The user interface 2500 may be displayed on the display device 2150 of FIG. 21.

FIG. 25B is an example user interface 2505 showing instructions for an SP procedure, according to various embodiments. The SP procedure may be an SP procedure for an endoscope. The instructions may include a description 2530 of a set of actions to be performed, or of parts, steps, or sub-steps of the SP procedure. In an example, the description 2530 includes a set of steps for processing an endoscope. The instructions may include one or more indicators 2532 associated with the actions to be performed or parts, steps, or sub-steps of the SP procedure. The one or more indicators 2532 may indicate a progress of the SP procedure and/or a completion of the actions to be performed or parts, steps, or sub-steps of the SP procedure.

The user interface 2505 may be interactive, such that selecting the one or more indicators 2532 causes the user interface 2505 to show more detailed instructions for the corresponding action to be performed, or part, step, or sub-step of the SP procedure. Selecting the one or more indicators 2522 may cause the user interface 2505 to provide more detailed instructions or explanations of the instruments, and/or the parts or locations of the instruments. Selecting the one or more indicators 2522 may cause the user interface 2505 to provide more detailed instructions for the corresponding action to be performed, or part, step, or sub-step of the SP procedure. In an example, selecting the one or more indicators 2522 causes a more detailed description of a step to be displayed. In this example, the more detailed description may include images, diagrams, or videos for the step.

The user interface 2505 may be displayed on a display device such as an interactive wall chart, a computer monitor, and/or an AR headset. The user interface 2505 may be displayed on the display device 2150 of FIG. 21.

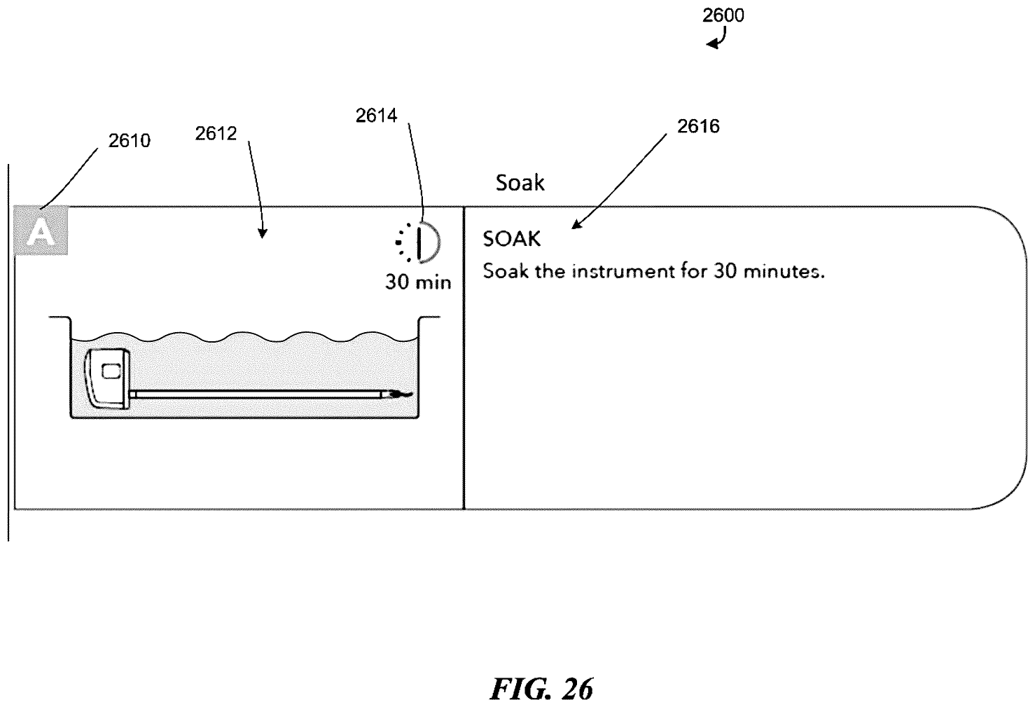

FIG. 26 is an example user interface 2600 showing instructions for an SP procedure, according to various embodiments. The instructions may include an identifier 2610 of a part, step, and/or sub-step of the SP procedure. The instructions may include a diagram 2612 showing an instrument soaking in a solution. The instructions may include a time indicator 2614 indicating how long the instrument should soak. The time indicator 2614 may be dynamic, starting a timer when the instrument is placed in the solution and indicating how much time is left to complete the soak. The instructions may include a description 2616 indicating an action to be performed, such as soaking the instrument for thirty minutes.

The user interface 2600 may be interactive, such that selecting the identifier 2610 causes the user interface 2600 to show the part, step, or sub-step within the SP procedure. Selecting the time indicator 2614 may start a timer and cause the time indicator 2614 to dynamically show a remaining amount of time for the instrument soak. Selecting the description 2616 may cause the user interface 2600 to display more a more detailed description, an image, or a video. Selecting the diagram 2612 may cause the diagram 2612 to be animated and/or may cause the user interface to display an image or video corresponding to the diagram 2612.

The user interface 2600 may be displayed on a display device such as an interactive wall chart, a computer monitor, and/or an AR headset. The user interface 2600 may be displayed on the display device 2150 of FIG. 21.

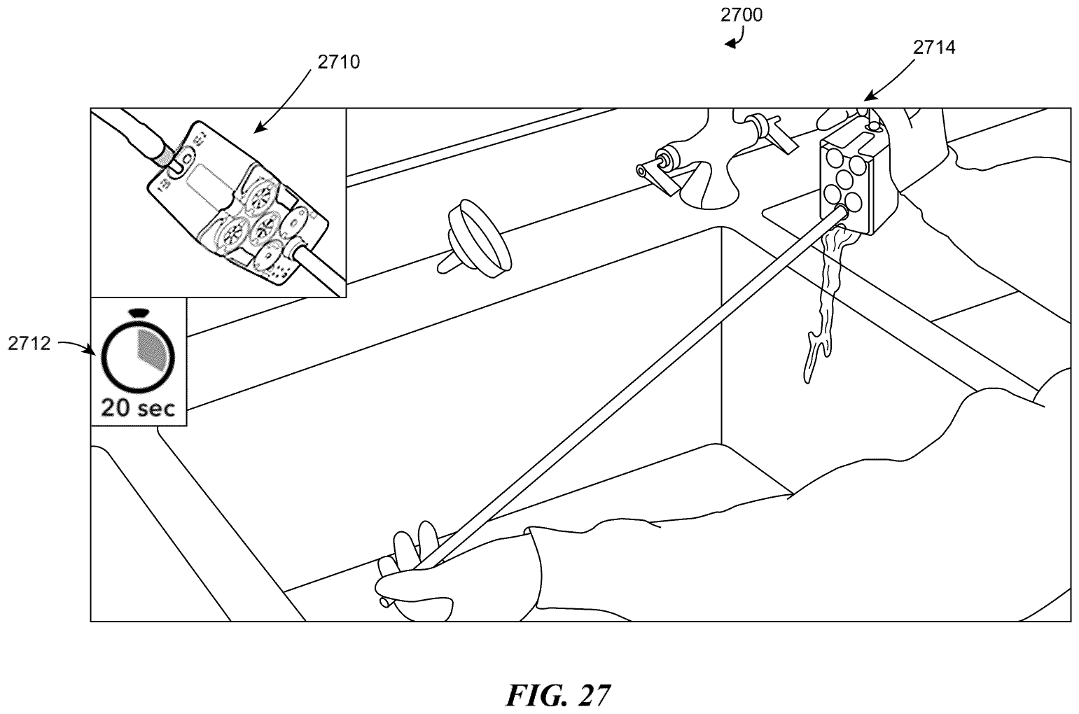

FIG. 27 is an example user interface 2700 showing instructions for an SP procedure, according to various embodiments. The instructions may include a diagram 2710 showing an instrument being connected to a water supply to flush the instrument. The instructions may include a time indicator 2712 indicating how long the instrument should be flushed. The time indicator 2712 may be dynamic, starting a timer when the instrument begins to be flushed and indicating how much time is left to complete the flush. The instructions may include an image or video 2714 demonstrating performance of the flush.

The user interface 2700 may be interactive, such that selecting the time indicator 2712 may start a timer and/or cause the time indicator 2712 to dynamically show a remaining amount of time for the instrument flush. Selecting the diagram 2710 may cause the diagram 2710 to be animated and/or may cause the user interface to display an image or video corresponding to the diagram 2710. Selecting the image or video 2714 may cause the image or video 2714 to be paused or cause subtitles to appear on the image or video 2714.

The user interface 2700 may be displayed on a display device such as an interactive wall chart, a computer monitor, and/or an AR headset. The user interface 2700 may be displayed on the display device 2150 of FIG. 21.

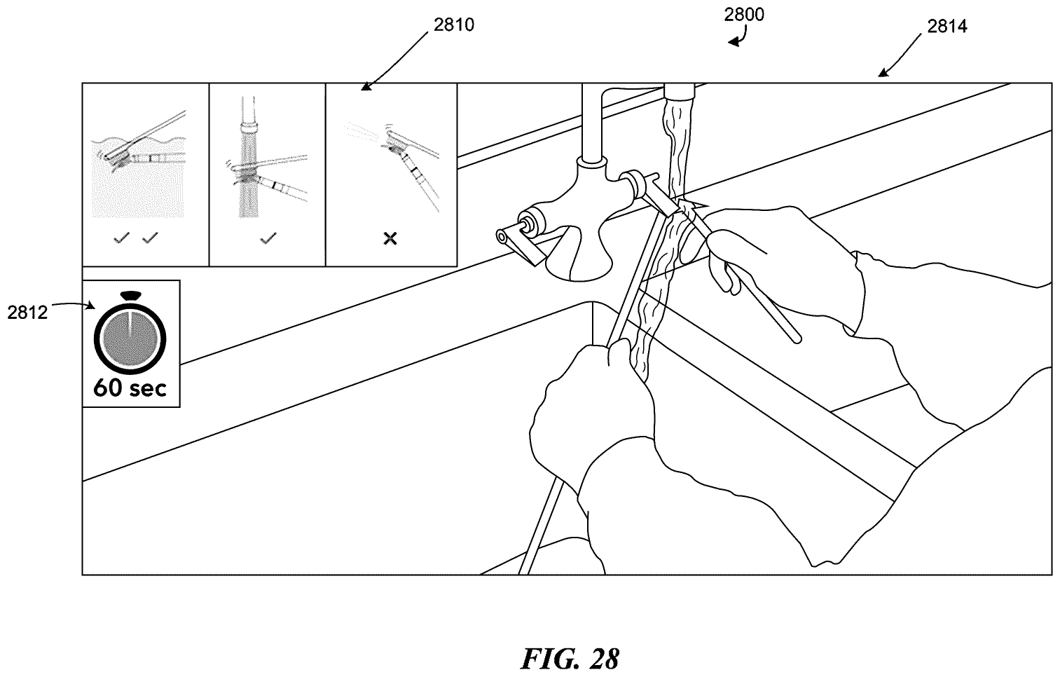

FIG. 28 is an example user interface 2800 showing instructions for an SP procedure, according to various embodiments. The instructions may include a diagram 2810 showing an instrument being scrubbed. The diagram 2810 may indicate correct and incorrect scrubbing methods. The diagram 2810 may indicate an incorrect scrubbing method, an acceptable scrubbing method, and a preferred scrubbing method. The instructions may include a time indicator 2812 indicating how long the instrument should be scrubbed. The time indicator 2812 may be dynamic, starting a timer when the instrument begins to be scrubbed and indicating how much time is left to complete the instrument scrub. The instructions may include an image or video 2814 demonstrating performance of the instrument scrub.

The user interface 2800 may be interactive, such that selecting the time indicator 2812 may start a timer and/or cause the time indicator 2812 to dynamically show a remaining amount of time for the instrument flush. Selecting the diagram 2810 may cause the diagram 2810 to be animated and/or may cause the user interface to display an image or video corresponding to the diagram 2810. Selecting the image or video 2814 may cause the image or video 2814 to be paused or cause subtitles to appear on the image or video 2814.

The user interface 2800 may be displayed on a display device such as an interactive wall chart, a computer monitor, and/or an AR headset. The user interface 2800 may be displayed on the display device 2150 of FIG. 21.

### Remarks

The drawings and description herein are illustrative. Consequently, neither the description nor the drawings should be construed so as to limit the disclosure. For example, titles or subtitles have been provided simply for the reader's convenience and to facilitate understanding. Thus, the titles or subtitles should not be construed so as to limit the scope of the disclosure, e.g., by grouping features which were presented in a particular order or together simply to facilitate understanding. Unless otherwise defined herein, all technical and scientific terms used herein have the same meaning as commonly understood by one of ordinary skill in the art to which this disclosure pertains. In the case of conflict, this document, including any definitions provided herein, will control. A recital of one or more synonyms herein does not exclude the use of other synonyms. The use of examples anywhere in this specification including examples of any term discussed herein is illustrative only and is not intended to further limit the scope and meaning of the disclosure or of any exemplified term.

Similarly, despite the particular presentation in the figures herein, one skilled in the art will appreciate that actual data structures used to store information may differ from what is shown. For example, the data structures may be organized in a different manner, may contain more or less information than shown, may be compressed and/or encrypted, etc. The drawings and disclosure may omit common or well-known details in order to avoid confusion. Similarly, the figures may depict a particular series of operations to facilitate understanding, which are simply exemplary of a wider class of such collection of operations. Accordingly, one will readily recognize that additional, alternative, or fewer operations may often be used to achieve the same purpose or effect depicted in some of the flow diagrams. For example, data may be encrypted, though not presented as such in the figures, items may be considered in different looping patterns (“for” loop, “while” loop, etc.), or sorted in a different manner, to achieve the same or similar effect, etc.

Reference herein to “an embodiment” or “one embodiment” means that at least one embodiment of the disclosure includes a particular feature, structure, or characteristic described in connection with the embodiment. Thus, the phrase “in one embodiment” in various places herein is not necessarily referring to the same embodiment in each of those various places. Separate or alternative embodiments may not be mutually exclusive of other embodiments. One will recognize that various modifications may be made without deviating from the scope of the embodiments.

What is claimed is:

1. A system, comprising:
one or more processors, coupled with memory, to:
receive, from one or more first sensors located in a decontamination room, multi-modal data comprising three-dimensional data of at least one sterile processing (SP) procedure performed in a decontamination room;
determine, using a first activity recognition machine-learning model, one or more SP actions based at least in part on the multi-modal data;
determine, using an SP analysis machine-learning model, an SP metric value based at least in part on the one or more SP actions, wherein the SP metric value is indicative of at least one of efficiency or efficacy of the at least one SP procedure; and
provide the SP metric value to a display device for display.

2. The system of claim 1, the one or more processors to determine, using a second activity recognition machine-learning model, one or more medical environment actions based on second multi-modal data including second three-dimensional data of an medical environment.

3. The system of claim 2, wherein the first activity recognition machine-learning model is trained using decontamination room data, and wherein the second activity recognition machine-learning model is trained using medical environment data.

4. The system of claim 1, wherein determining the one or more SP actions includes adding timestamps to the three-dimensional data associated with the one or more SP actions.

5. The system of claim 1, wherein the SP analysis machine-learning model uses as input robotic system data corresponding to medical procedures using instruments for which SP is performed.

6. The system of claim 5, wherein the robotic system data indicates usage of the instruments.

7. The system of claim 5, wherein the SP analysis machine-learning model receives the robotic system data from a robotic system in an medical environment.

8. The system of claim 7, wherein the SP analysis machine-learning model tracks the instruments from usage in the medical environment to completion of the at least one SP procedure in the decontamination room.

9. The system of claim 1, wherein the SP metric value includes or is determined based at least in part on an SP turnaround time.

10. The system of claim 9, wherein the SP metric value includes or is determined based at least in part on SP turnaround times for each medical procedure of a plurality of medical procedures.

11. The system of claim 9, wherein the SP metric value includes or is determined based at least in part on SP turnaround times for each instrument type of a plurality of instrument types.

12. The system of claim 1, wherein the SP metric value includes one or more of SP equipment utilization, completion of steps of the at least one SP procedure, temporal metrics for the steps of the at least one SP procedure, and SP throughput.

13. The system of claim 1, the one or more processors to generate, using the SP analysis machine-learning model, alerts based on detecting one or more of detecting mishandled instruments, contamination of instruments, and infection risk for SP staff.

14. The system of claim 1, the one or more processors to generate one or more recommendations based on one or more of the SP metric value and the three-dimensional data of the at least one SP procedure.

15. The system of claim 14, wherein the one or more recommendations include one or more of training for SP staff, decontamination room layout optimization, better resource allocation to minimize equipment idle time, and optimized staff and equipment levels.

16. The system of claim 1, wherein the display device is an interactive display.

17. The system of claim 1, wherein the display device indicates a status of instruments being processed.

18. The system of claim 1, wherein the display device includes an AR headset.

19. The system of claim 1, wherein the display device includes information, videos, images, or guidance related to performing an identified part, step, or sub-step along with an SP metric value for that part, step, or sub-part.

20. The system of claim 1, wherein the one or more first sensors include egocentric and exocentric sensors.

21. A system, comprising:
one or more processors, coupled with memory, to:
receive, from one or more first sensors located in a decontamination room, multi-modal data comprising three-dimensional data of at least one sterile processing (SP) procedure performed in the decontamination room, wherein the SP procedure comprises sterilizing an instrument used in a medical procedure performed in a medical environment or an instrument coupled to a robotic system for performing the medical procedure in the medical environment;
determine, using a first activity recognition machine-learning model, one or more SP actions based at least in part on the multi-modal data;
determine, using an SP analysis machine-learning model, an SP metric value for the one or more SP actions, wherein the SP metric value is determined based at least in part on a length of time of at least a portion of the one or more SP actions; and
provide the SP metric value to a display device for display.

22. The system of claim 21, the one or more processors to determine, using a second activity recognition machine-learning model, one or more medical environment actions based on second multi-modal data including second three-dimensional data of an medical environment.

23. The system of claim 22, wherein the first activity recognition machine-learning model is trained using decontamination room data, and wherein the second activity recognition machine-learning model is trained using medical environment data.

24. The system of claim 21, wherein the SP analysis machine-learning model uses as input robotic system data corresponding to medical procedures using instruments for which SP is performed.

25. The system of claim 21, wherein the SP metric value includes one or more of SP equipment utilization, completion of steps of the at least one SP procedure, temporal metrics for the steps of the at least one SP procedure, and SP throughput.

26. The system of claim 21, the SP analysis machine-learning model to generate alerts based on detecting one or more of detecting mishandled instruments, contamination of instruments, and infection risk for SP staff.

27. The system of claim 21, the one or more processors to generate one or more recommendations based on one or more of the SP metric value and the three-dimensional data of the at least one SP procedure.

28. A system, comprising:
one or more processors, coupled with memory, to:
receive a plurality of streams of data of a sterile processing (SP) procedure performed in a decontamination room, wherein the plurality of streams of data comprise three-dimensional data of the SP procedure;
determine, using a machine-learning model, an SP metric value for at least a portion of the SP procedure based at least in part on the plurality of streams of data of the SP procedure; and
provide the SP metric value to be displayed on a user interface.

29. The system of claim 28, the one or more processors to determine, using a second machine-learning model, one or more medical environment actions based on second multi-modal data including second three-dimensional data of an medical environment.

30. The system of claim 29, wherein the machine-learning model is trained using decontamination room data, and wherein the second machine-learning model is trained using medical environment data.

31. The system of claim 28, wherein the machine-learning model uses as input robotic system data corresponding to medical procedures using instruments for which SP is performed.

32. The system of claim 28, wherein the SP metric value includes or is determined based at least in part on an SP turnaround time.

33. The system of claim 32, wherein the SP metric value includes or is determined based at least in part on an SP turnaround time for a medical procedure of a plurality of medical procedures.

34. The system of claim 32, wherein the SP metric value includes or is determined based at least in part on an SP turnaround time for an instrument type of a plurality of instrument types.

35. The system of claim 28, wherein the SP metric value includes one or more of SP equipment utilization, completion of steps of the SP procedure, temporal metrics for the steps of the at least one SP procedure, and SP throughput.

36. The system of claim 28, the machine-learning model to generate alerts based on detecting one or more of detecting mishandled instruments, contamination of instruments, and infection risk for SP staff.

37. The system of claim 28, the one or more processors to generate one or more recommendations based on one or more of the SP metric value and the three-dimensional data of the SP procedure.
